SEQUENCE LISTING (4 sequences)

SEQ ID NO: 1 - DNA, 4716 bp, Streptococcus agalactiae
atgaacacaaaacagcgtttttcaatccggaaatataagttaggtgccgtatctgtacttttgggaaccctattttttttaggcggtatcacaaatgtagctgctgattctgtcataaataagccatctgatattgcagttgaacagcaagtaaaagacagtccaacgagcatagcaaatgagacacctactaacaacacgtcatcagcccttgcgtcaacagctcaagacaatcttgttacaaaggctaataataatagtccaacagaaacacaaccagtaactgagcctcactctcaagccaccgagacattttccccagccgcaaatcaaccggttgaaagcactcaagaagtttctaaaactcctttaaccaaacaaaatttagcagtcaaacctacaccagctatttctaaagaaacccctcaaaacattgatagtaataaaattatcactgtccccaaagtatggaacacaggctacaaaggagagggaactgttgtagcaattattgactcaggactagataccaatcacgatgctctccaattaaatgattcgacaaaagcaaaataccaaaacgaacagcaaatgaatgctgctaaagcaaaagctggtataaactatggaaaatggtataacaacaaagtaatctttggtcacaactatgttgatgtcaatacagagctaaaagaggtgaaaagcacttctcatggtatgcacgtaaccagtatcgcaacagctaatcctagcaagaaagatacaaatgaattaatctatggtgttgctcctgaagcacaagtaatgtttatgagagtcttctctgatgaaaaaagaggaactggaccagccctttatgttaaagctattgaagatgccgttaaactcggtgctgatagcattaatttaagtttaggtggagctaatggctctttagttaatgccgatgaccgacttataaaagctttagagatggctagactcgctggcgtttctgttgttatagcagcaggtaacgacggtacatttgggagtggagcatcaaagccttctgctctttatcctgattatggtttagttggtagtccatcaacagctcgtgaggccatttctgtagcatcatataataatacaacactggttaataaagtcttcaacattatcggattagaaaacaacaaaaatctcaacaacggattagctgcttatgcagatcctaaagttagtgataagacctttgaagtagggaagcaatatgattatgttttcgtaggaaaaggaaacgacaatgattataaggacaaaactttaaatggtaaaatcgccttaattgaacgtggagatattacttttacaaaaaaagtcgtcaacgctattaatcacggtgctgtgggagctattatctttaataacaaagctggagaagctaatctaacaatgagtttagatcctgaagcaagcgctattcctgccatttttactcaaaaagagtttggagatgttttagctaaaaacaactataaaattgtatttaacaatatcaaaaataaacaagccaaccctaatgcaggtgtcctatctgacttttcaagctggggattaacagcagacggacaattaaaacctgacttatctgctcctggaggctctatttacgccgctatcaatgataatgaatatgatatgatgagtgggacaagtatggcttctccccatgtcgctggtgctactgctctagttaaacaatacttattgaaagaacatccagaacttaaaaaaggtgacattgaaagaactgtcaaataccttcttatgagtactgctaaagcacacctaaacaaagatacaggcgcttacacctcaccacgccaacaaggagcaggtattatcgatgtcgcagcagcagttcagacaggattatacctaactggtggggaaaacaactatggtagcgttacattaggaaatattaaagataaaatttcctttgatgttactgttcataatatcaataaagttgcaaaagatttacactatacaacctatttaaatactgatcaagttaaagatggctttgtcacattagctcctcaacaacttggtacatttacagggaaaacgatacggattgaaccagggcaaactaaaacgattacaattgatatagatgtttcgaaataccatgacatgttaaaaaaagtaatgccaaacggctatttcctagaaggctacgtacgttttacagaccctgttgatggtggggaagttcttagtattccttatgttggatttaagggagaattccaaaacttagaagttttagaaaaatccatttataagcttgttgctaacaaagaaaagggattttatttccaaccgaaacaaacaaacgaagttcctggttcagaagattatactgccttaatgactacaagttcagagcctatctactcaacagacggtactagtcctatccaattgaaagccttgggaagctataagtctatagatggaaaatggatcttacaactagagcaaaaaggccagcctcatctagccatttcacctaatgatgaccaaaatcaagatgccgttgcactgaaaggtgttttcttacgtaatttcaataatttaagagccaaagtctatcgtgcagatgatgttaatttacaaaaaccactatgggtaagtgctccccaagcaggagataaaaattactacagcggaaatactgaaaatccaaaatctacatttttatatgacacagaatggaaaggaaccactactgatggtattcctttagaagatggaaaatacaaatacgttttaacatactactctgatgtccctggctctaagccacaacaaatggtatttgatatcactttggatagacaagctcctacactaacaacagcaacttatgacaaagatagacgtatcttcaaagctcgtcctgcagtagaacacggggaatctggtatctttagagaacaagttttttacttaaaaaaagataaagatggtcattataatagcgtcttacgtcaaaaaggagaagacggtatccttgttgaagataacaaagtatttatcaaacaagaaaaggatggtagctttattctacctaaagaggttaatgatttctctcatgtctactacactgttgaagattatgcaggcaatctagtgtcagcaaaactcgaagatttgatcaatattggcaataaaaatggtttagtaaacgtcaaagtgtttagccctgagcttaacagtaatgtcgatattgatttctcttactctgtcaaagatgacaaaggtaatgtcatcaaaaagcaacatcacgggaaagacctcaatttactgaaattgccttttggtacctatacgtttgacctattcttatacgatgaggaacgagcaaatctaatcagtccccaaagtgtcactgtaactatttctgaaaaagatagccttaaagacgtcttatttaaagttaacttactcaagaaagcagccttactcgttgaatttgacaagcttttaccaaaaggagcaacagtccagttggttactaagacaaatactgttgttgatctaccaaaagcaacttattctcctactgactatggtaaaaacatacctgtaggagactatcgtttaaacgtaacgctgcctagtgggtatagcactttagagaacttagatgatttacttgtatccgtaaaagaaggtcaagtaaatctaacaaaattgacgctgattaataaagctcctctaattaatgccctagcagaacaaactgacattatttcccaacctgtgttttataatgctggaactcacttaaaaaataattacctagctaatcttgaaaaggcacaaactttaattaaaaatagagtggaacaaacaagtattgataatgctattgctgctttgagagaaagtcgccaagctcttaacggtaaagaaacagatacttctttactggcaaaagctattttagctgaaacagaaatcaagggaaactatcaatttgttaatgctagtccattaagccaatcaacttatatcaatcaagtccaattggcgaaaaatcttctacaaaaacctaacgtcactcaatcagaagtagacaaagccttagaaaatcttgatattgctaaaaatcaattaaatggtcatgaaactgattactctggtttacaccatatgataattaaagcaaatgttctgaaacaaacatcatctaaatatcagaacgccagtcaatttgctaaagaaaattataataaccttatcaagaaagcagaattgctgctttccaatagacaagctacacaagctcaagttgaagagttattaaaccaaataaaagcaaccgaacaagaacttgatggtcgcgatagagtttcttccgcagagaattatagtcaatcactcaatgataatgactctctcaataccacacctatcaatccgccaaatcagccccaggcgttgatattcaaaaaaggcatgactaaagaaagtgaggttgctcagaagcgtgtcttaggggtgactagccaaaccgataatcaaaagataaagacaaacaagcttcctaaaacaggcgaaagcactcctaaaataacctatacaatattgctatttagtctctctatgctaggtctggcaacaatcaaactaaagtctatcaaaagagaataa

SEQ ID NO: 2 - protein, 1571 aa, Streptococcus agalactiae
MNTKQRFSIRKYKLGAVSVLLGTLFFLGGITNVAADSVINKPSDIAVEQQVKDSPTSIANETPTNNTSSALASTAQDNLVTKANNNSPTETQPVTEPHSQATETFSPAANQPVESTQEVSKTPLTKQNLAVKPTPAISKETPQNIDSNKIITVPKVWNTGYKGEGTVVAIIDSGLDTNHDALQLNDSTKAKYQNEQQMNAAKAKAGINYGKWYNNKVIFGHNYVDVNTELKEVKSTSHGMHVTSIATANPSKKDTNELIYGVAPEAQVMFMRVFSDEKRGTGPALYVKAIEDAVKLGADSINLSLGGANGSLVNADDRLIKALEMARLAGVSVVIAAGNDGTFGSGASKPSALYPDYGLVGSPSTAREAISVASYNNTTLVNKVFNIIGLENNKNLNNGLAAYADPKVSDKTFEVGKQYDYVFVGKGNDNDYKDKTLNGKIALIERGDITFTKKVVNAINHGAVGAIIFNNKAGEANLTMSLDPEASAIPAIFTQKEFGDVLAKNNYKIVFNNIKNKQANPNAGVLSDFSSWGLTADGQLKPDLSAPGGSIYAAINDNEYDMMSGTSMASPHVAGATALVKQYLLKEHPELKKGDIERTVKYLLMSTAKAHLNKDTGAYTSPRQQGAGIIDVAAAVQTGLYLTGGENNYGSVTLGNIKDKISFDVTVHNINKVAKDLHYTTYLNTDQVKDGFVTLAPQQLGTFTGKTIRIEPGQTKTITIDIDVSKYHDMLKKVMPNGYFLEGYVRFTDPVDGGEVLSIPYVGFKGEFQNLEVLEKSIYKLVANKEKGFYFQPKQTNEVPGSEDYTALMTTSSEPIYSTDGTSPIQLKALGSYKSIDGKWILQLEQKGQPHLAISPNDDQNQDAVALKGVFLRNFNNLRAKVYRADDVNLQKPLWVSAPQAGDKNYYSGNTENPKSTFLYDTEWKGTTTDGIPLEDGKYKYVLTYYSDVPGSKPQQMVFDITLDRQAPTLTTATYDKDRRIFKARPAVEHGESGIFREQVFYLKKDKDGHYNSVLRQKGEDGILVEDNKVFIKQEKDGSFILPKEVNDFSHVYYTVEDYAGNLVSAKLEDLINIGNKNGLVNVKVFSPELNSNVDIDFSYSVKDDKGNVIKKQHHGKDLNLLKLPFGTYTFDLFLYDEERANLISPQSVTVTISEKDSLKDVLFKVNLLKKAALLVEFDKLLPKGATVQLVTKTNTVVDLPKATYSPTDYGKNIPVGDYRLNVTLPSGYSTLENLDDLLVSVKEGQVNLTKLTLINKAPLINALAEQTDIISQPVFYNAGTHLKNNYLANLEKAQTLIKNRVEQTSIDNAIAALRESRQALNGKETDTSLLAKAILAETEIKGNYQFVNASPLSQSTYINQVQLAKNLLQKPNVTQSEVDKALENLDIAKNQLNGHETDYSGLHHMIIKANVLKQTSSKYQNASQFAKENYNNLIKKAELLLSNRQATQAQVEELLNQIKATEQELDGRDRVSSAENYSQSLNDNDSLNTTPINPPNQPQALIFKKGMTKESEVAQKRVLGVTSQTDNQKIKTNKLPKTGESTPKITYTILLFSLSMLGLATIKLKSIKRE

SEQ ID NO: 3 - DNA, 23 bp, Artificial Sequence
  primer
tcggatccgctactgctctagtt

SEQ ID NO: 4 - DNA, 29 bp, Artificial Sequence
  primer
ttaagtcgacgtaatgatgccttgctcta

(19) United States
(12) Patent Application Publication
(10) Pub. No.: US 2005/0002950 A1
(43) Pub. Date: Jan. 6, 2005

(54) Use of a novel cell surface protease from Group B Streptococcus

(76) Inventors: Craig E. Rubens, Seattle, WA (US); Theresa Harris, Seattle, WA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/864,138

(22) Filed: Jun. 9, 2004

Related U.S. Application Data
(63) Continuation of application No. PCT/US02/40340, filed on Dec. 16, 2002.
(60) Provisional application No. 60/344,605, filed on Dec. 21, 2001.

(51) Int. Cl. A61K 39/00; A61K 39/38

(52) U.S. Cl. 424/184.1

(57) ABSTRACT

This invention relates to use of a novel cell surface protease protein of Group B Streptococcus (GBS), called CspA, as a vaccine to prevent GBS infection.

### RELATED APPLICATIONS

This application is a continuation of International Patent Application No. PCT/US02/40340, filed Dec. 16, 2002 designating the United States of America and published in English on Jul. 24, 2003 as WO 03/059252, which claims the benefit of priority of U.S. Provisional Application No. 60/344,605 filed Dec. 21, 2001, both of which are hereby expressly incorporated by the reference in their entireties.

### GOVERNMENT RIGHTS

The U.S. Government has a paid-up license in this invention and the right in limited circumstances to require the patent owner to license others on reasonable terms as provided for by the terms of Grant No. A130068 awarded by the National Institutes of Health.

### FIELD OF THE INVENTION

This invention relates to use of a novel cell surface protease protein of Group B Streptococcus (GBS), called CspA, as a vaccine to prevent GBS infection.

### BACKGROUND OF THE INVENTION

Group B Streptococcus (GBS), also known as Streptococcus agalactiae, is the causative agent of various conditions. In particular, GBS causes:

Early Onset Neonatal Infection.

This infection usually begins in utero and causes severe septicemia, pneumonia, and meningitis in infants, which is lethal if untreated and even with treatment is associated with a 10-20% mortality rate.

Late Onset Neonatal Infection.

This infection occurs in the period shortly after birth until about 3 months of age. It causes a septicemia, which is complicated by meningitis in 90% of cases. Other focal infections also occur including osteomyelitis, septic arthritis, abscesses and endopthalmitis.

Adult Infections.

These appear to be increasingly common and occur most frequently in women who have just delivered a baby, the elderly and the immunocompromised. They are characterized by septicemia and focal infections including osteomyelitis, septic arthritis, abscesses and endopthalmitis.

Urinary Tract Infections.

GBS is a cause of urinary tract infections and in pregnancy accounts for 10% of all infections.

Veterinary Infections.

GBS causes chronic mastitis in cows. This, in turn, leads to reduced milk production and is therefore of considerable economic importance.

GBS infections can be treated with antibiotics. However, immunization is preferable. It is therefore desirable to develop an immunogen that could be used in a therapeutically effective vaccine.

### SUMMARY OF THE INVENTION

This invention relates to use of a novel cell surface protease protein of Group B Streptococcus (GBS), called CspA, as a vaccine to prevent GBS infection.

### BRIEF DESCRIPTION OF THE DRAWINGS

FIG. 1A-C is the complete nucleotide coding sequence for CspA.

FIG. 2 is the complete amino acid coding sequence for CspA.

FIG. 3 is a map of the cspA region in type III Group B Streptococcus (GBS). Arrows depict the identified open reading frames (ORFs) and their direction of transcription. Restriction endonuclease recognition site designations are C, ClaI; X, XbaI, and H, HindIII. Plasmid inserts used to sequence the locus are depicted above the map. The terminal 5′ and 3′HindIII sites at positions 2342 and 5983 were used to replace the internal cspA coding sequence with erm^{r}. sbxA encodes a product of 202 aa with no homology to characterized proteins. The 337 aa product of sbtA shares significant homology to the C-terminal coding region of numerous tRNA synthetases. Shown below the ORF map are protein motifs identified in the predicted sequence of CspA (Siezen, R. J. 1999 Antonie Van Leeuwenhoek 76:139-155); signal sequence (SS; residues 1-35), pre-pro domain (36-143), protease domain (144-638), A domain (639-1076), cell wall spacer domain (1077-1535) and cell wall anchor domain (CWA; 1536-1571). An RGD motif (D'Souza, S. E. et al. 1991 Trends Biochem Sci 16:246-250) is present in the protease domain and position 446. The function of the A domain is unknown, but it may be involved in regulation or substrate specificity of the protease domain (Siezen, R. J. 1999 Antonie Van Leeuwenhoek 76:139-155).

FIG. 4 shows a Northern blot analysis of cspA expression in COH1 and TOH121. Standard Northern blots were performed using equivalent amounts of RNA (5 μg) from the indicated strains. A. Blot developed using cspA probe. Lane 1, COH1 grown to OD₆₀₀=0.3 in THB, lane 2, COH1 OD=0.6; lane 3, COH1 OD=1.7; lane 4, TOH121 at OD=0.3; and lane 5, TOH121 at OD=1.7. B. Blot developed using sbrA probe. Lane 1, COH1 at OD=0.6; lane 2, TOH121 at OD=0.6. Migration of RNA size standards is indicated (in kb) on the right.

FIG. 5 shows a Western blot analysis of CspA expression in E. coli and GBS strains using anti-CspA sera. Lane 1, periplasmic extract from E. coli DH5α containing pBS (negative control); Lane 2, periplasmic extract from E. coli DH5α with pTH5 (XbaI fragment bearing cspA in pBSKS- (Stratagene, La Jolla, Calif., USA). Note that the lower migration of CspA in periplasmic extracts is due to a mutation in pTH5 that prematurely terminates translation (see below); lanes 3-6, mutanolysin-extracted GBS surface proteins from COH1 (lane 3), TOH121 (cspA⁻; lane 4), TOH97 (scpB⁻; lane 5) and TOH144 (cspA⁻ scpB⁻; lane 6). Molecular mass markers are indicated on the left.

FIG. 6 shows functional assay for C5a protease activity. Shown is the percent adhesion by human polymorphonuclear leukocytes (PMN) to gelatin-coated tissue culture wells after the indicated GBS strains were incubated with recombinant human C5a (Bohnsack, J. F. et al. 1991 Biochem J 273:635-640). As controls, buffer alone (‘buffer’) or 100 ng per ml of untreated C5a (‘C5a’) were incubated without bacteria before exposure to PMN. GBS strains that were tested were COH1 (positive control; cspA⁺, scpB⁺), GW (negative control, a type III GBS strain lacking C5a-ase activity (Bohnsack, J. F. et al. 2000 Infect Immun 68:5018-5025)), TOH97 (negative control; cspA⁺, scpB⁻), TOH121 (cspA⁻, scpB⁺), TOH144 (cspA⁻, scpB⁻).

FIG. 7 shows caseinase activity assay. GBS strain COH1 was grown to stationary phase in Todd-Hewitt Broth (THB), washed in phosphate buffered saline (PBS), concentrated 20-fold, and resuspended in PBS. A saturated solution of protease-assay grade casein (Sigma, St. Louis, Mo.) was prepared, and 0.2 ml of bacteria was mixed with 0.3 ml casein. A mock reaction with PBS (no bacteria) was also prepared. The mixtures were incubated for 24 hours at 37 C, and an aliquot was removed for sodium dodecyl sulphate polyacrylamide gel electrophoresis (SDS-PAGE). Molecular weight markers are depicted on the left. Lane 1, PBS control; Lane 2, COH1 after 0 hr incubation, Lane 3, COH1 after 24 hr incubation. Migration of molecular mass markers (in kDa) is indicated on the left.

FIG. 8 shows fibrinogen degradation assay. GBS strains COH1 (wt) and TOH121 (cspA⁻) were grown to stationary phase in THB. Cells were washed once in an equivalent volume of PBS, and concentrated 20-fold. Fibrinogen was then added to a concentration of 0.61 ug/ml. The suspension was incubated with slow rotation overnight (16 hr) at 37° C., bacteria were removed by centrifugation, and the supernatant, containing 3 μg total fibrinogen, was analyzed by SDS-PAGE as follows: Lane 1, COH1 after 0 hr incubation; Lane 2, TOH121 after 0 hr incubation; Lane 3, COH1 after 16 hr incubation; Lane 4, TOH121 after 16 hr incubation. The arrow at the left of the figure denotes the migration position of the minor species of the α fragment of fibrinogen that is proteolyzed by CspA. Migration of molecular mass markers (in kDa) is indicated on the right.

FIG. 9 shows opsonophagocytosis of GBS strains by PMN. GBS strains COH1, TOH121, and the unencapsulated mutant COH 1-13 were compared for resistance to opsonophagocytosis by PMN. Bacteria (1×10⁶) and human PMN (3×10⁶) were incubated in 10% human sera as a complement source (preabsorbed with COH1 to remove GBS Ab) for 1 h at 37° C. Growth index was calculated as the output colony-forming unit (CFU) per ml divided by input CFU per ml. One representative experiment of four is depicted. Controls were performed for the test strain, TOH121, with heat-inactivated serum (‘HIS’) and without PMN (‘-PMN’).

FIG. 10 is the number and numbering of residues per CspA domains.

FIG. 11 is the schematic representation of the predicted domains in CspA protease. PP=pre-pro domain; PR=protease domain; I=insert domain; A=A−domain; H=helical domain; W=cell-wall domain; AN=anchor domain (black dot indicates LPXTG motif, amino acids 1536-1540 of SEQ ID NO: 2).

### DETAILED DESCRIPTION OF THE PREFERRED EMBODIMENT

### A Novel Serine Protease-like Gene (cspA) Expressed by all Group B Streptococcal Serotypes is Important for Streptococcal Virulence

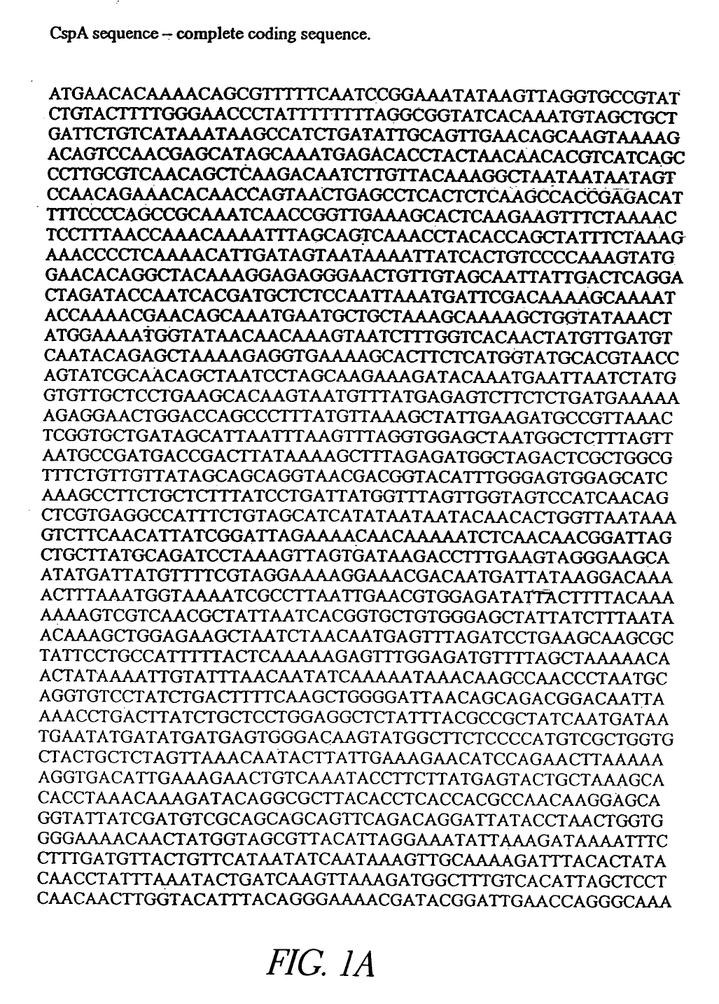

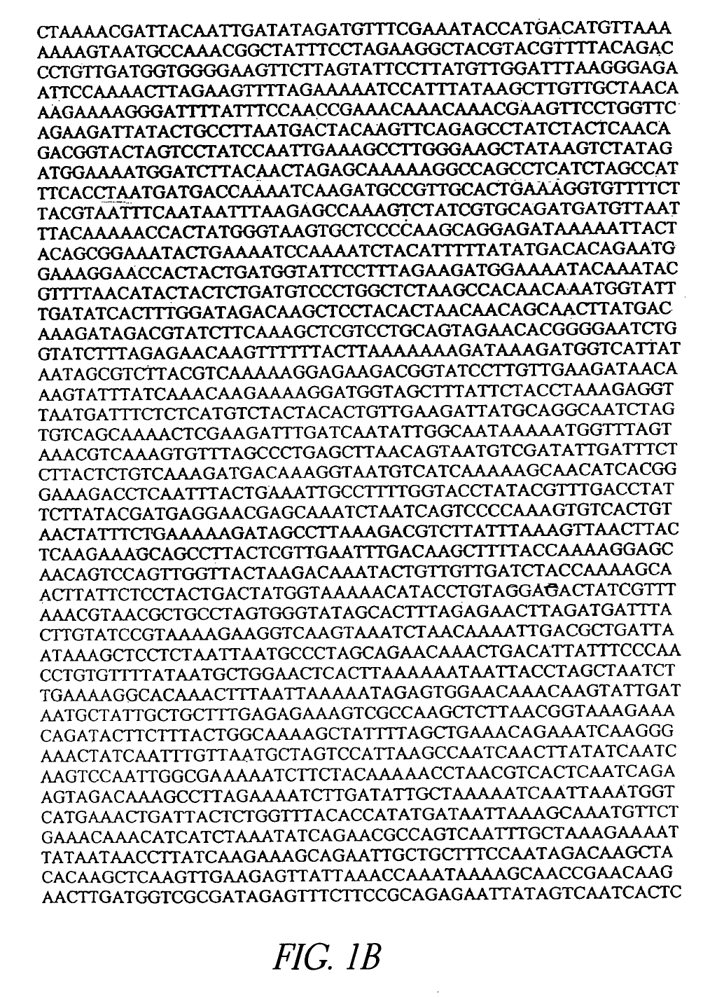

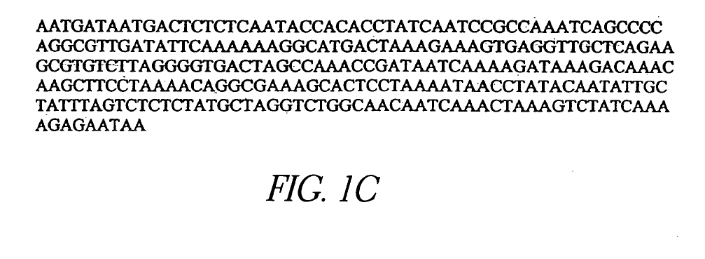

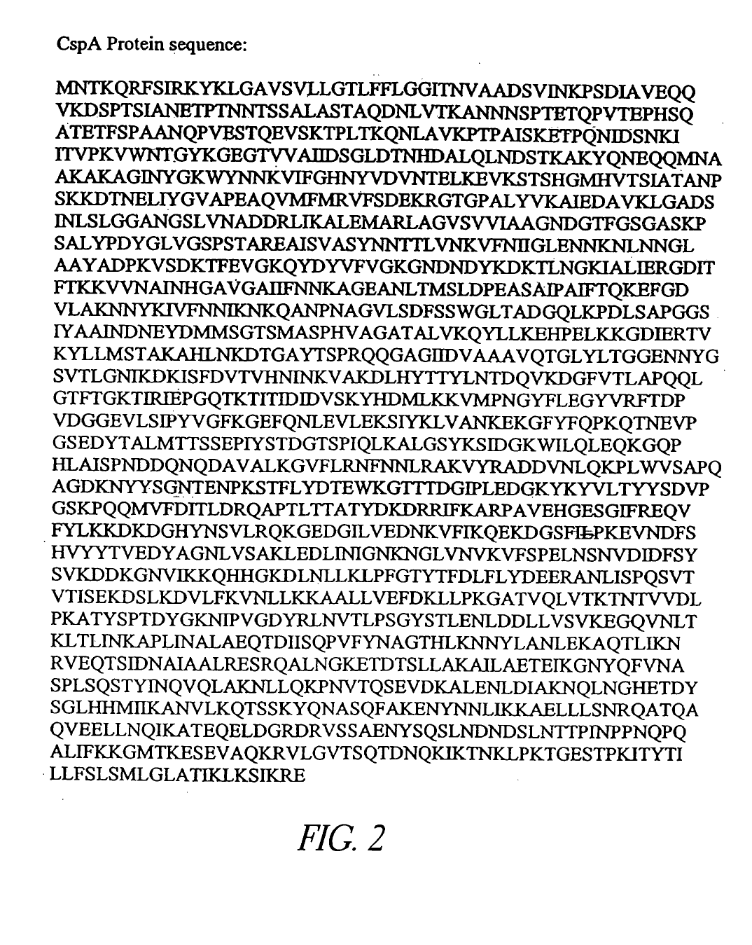

Group B Streptococcus (GBS) is an important human pathogen. In this study we sought to identify mechanisms that may protect GBS from host defenses in addition to its capsular polysaccharide. A gene encoding a cell-surface associated protein (cspA) (FIG. 1; SEQ ID NO: 1) was characterized from a highly virulent type III GBS isolate, COH1. Its sequence (FIG. 2; SEQ ID NO: 2) indicated that it is a subtilisin-like extracellular serine protease homologous to streptococcal C5a peptidases and caseinases of lactic acid bacteria. The wild-type strain cleaved the alpha chain of human fibrinogen, while a cspA mutant, TOH121, was unable to cleave fibrinogen. We observed aggregated material when COH1 was incubated with fibrinogen, but not when the mutant strain was treated similarly. This indicated that the product(s) of fibrinogen cleavage have strong adhesive properties and may be similar to fibrin. The cspA gene was present among representative clinical isolates from all nine capsular serotypes, as revealed by Southern blotting. A cspA⁻ mutant was ten-fold less virulent in a neonatal rat sepsis model of GBS infections, as measured by LD₅₀ analysis. Additionally, the cspA⁻ mutant was significantly more sensitive than the wild type strain to opsonophagocytic killing by human neutrophils in vitro. Taken together, the results indicate that cleavage of fibrinogen by CspA can increase the lethality of GBS infection by protecting the bacterium from opsonophagocytic killing.

Introduction.

Group B Streptococcus (GBS) is a major cause of serious neonatal bacterial infections (Baker, C. J. & Edwards, M. S. 1995 in: Infectious Diseases of the Fetus and Newborn Infant J. S. Remington & J. O. Klein, eds. Philadelphia: W. B. Saunders 980-1054) and is the most common cause of sepsis and pneumonia in newborns. GBS is also a significant etiology of postpartum endometritis. Additionally, recent data implicate GBS as an increasing cause of invasive infections in adults, especially among the immunocompromised (Farley, M. M. et al. 1993 N Engl J Med 328:1807-1811).

Bacterial pathogens have evolved a diverse array of defenses to combat the innate immune system, which is an important first line of defense against bacterial infections in the non-immune host. Complement as well as phagocytic cells such as polymorphonuclear leukocytes (PMN) and macrophages play major roles in innate immunity. The antiphagocytic mechanisms of a number of gram-negative bacteria such as Yersiniae and Pseudomonas aeruginosa (Ernst, J. 2000 Cellular Microbiology 2:379-386) are well characterized. However, much remains to be understood about how gram-positive pathogens evade phagocytic mechanisms. Streptococcus pneumoniae strains produce polysaccharide capsules (Dillard, J. P. et al. 1995 J Exp Med 181:973-983) that allow this bacterium to resist complement deposition in the absence of type-specific capsule antibodies. Additionally, S. pneumoniae strains produce a C3 binding protein that may function in the evasion of complement-mediated host defense (Cheng, Q. et al. 2000 Biochemistry 39:5450-5457). Enterococcus faecalis, a major cause of hospital-acquired infections, has recently been reported to synthesize a capsular polysaccharide that provides resistance to opsonophagocytic killing (Hancock, L. E. & Gilmore, M. S. 2002 PNAS USA 99:1574-1579). Group A Streptococcus (GAS) strains utilize numerous mechanisms to evade this immune pathway, including the M protein (Ashbaugh, C. et al. 1998 J Clin Invest 102:550-560) and the hyaluronic acid capsule (Wessels, M. R. et al. 1991 PNAS USA 88:8317-8321).

The GBS capsule is the most well-defined virulence factor of GBS. The capsule protects GBS from opsonization by C3 via inhibition of the alternative complement pathway in the absence of type specific capsule antibodies (Rubens, C. E. et al. 1987 PNAS USA 84:7208-7212). However, GBS may express additional factors that allow it to resist opsonophagocytosis. A recent report indicated that the surface-localized streptococcal protein binds human complement factor H, and that the GBS-factor H complex retains its ability to down-regulate complement activation (Areschoug, T. et al. 2002 J Biol Chem 277:12642-12648).

The present invention describes the identification of cspA, a novel surface-localized serine protease-like gene (cspA) that promotes GBS survival by evasion of opsonophagocytosis. CspA shows homology to a family of proteases that include C5a proteases of pathogenic streptococci (Bohnsack, J. F. et al. 1991 Biochim Biophys Acta 1079:222-228) as well as caseinases expressed by non-pathogenic Gram-positive cocci (Fernandez-Espla, M. D. et al. 2000 Appl Environ Microbiol 66:4772-4778). Surprisingly, we observed that CspA does not have enzymatic activity against C5a in vitro and the presence of the cspA gene was not required for casein degradation. However, the cspA gene was required for GBS cleavage of human fibrinogen, indicating that CspA is active as a protease. Mutants that failed to express cspA significantly decreased GBS virulence in a neonatal rat model of infection and displayed increased sensitivity to opsonophagocytosis. Our findings provide evidence that CspA is a novel surface-localized protease that plays an important role in GBS pathogenesis as an antiphagocytic surface factor.

Bacterial Strains.

COH1 is a highly encapsulated type III GBS strain, originally isolated from the blood of a septic newborn (Martin, T. R. et al. 1988 J Infect Dis 157:91-100). Other GBS clinical strains used in this work included: type 1a strains B523 (Tamura, G. S. & Rubens, C. E. 1995 Mol Microbiol 15:581-589), A909 (Tamura, G. S. & Rubens, C. E. 1995 Mol Microbiol 15:581-589), ChanS5; type Ib strains DK14, DK15, 80-481; type II strains 78-471 (Chmouryguina, I. et al. 1996 Infect Immun 64:2387-2390) and DK23 (Tamura, G. S. & Rubens, C. E. 1995 Mol Microbiol 15:581-589); type III strains COH31 (Rubens, C. E. et al. 1987 PNAS USA 84:7208-7212), D136C (Tamura, G. S. & Rubens, C. E. 1995 Mol Microbiol 15:581-589), M781 (Tamura, G. S. & Rubens, C. E. 1995 Mol Microbiol 15:581-589); type IV strain CNCTC1/82 (Tamura, G. S. & Rubens, C. E. 1995 Mol Microbiol 15:581-589), type V strains B201 and CNCTC 10/84 (Tamura, G. S. & Rubens, C. E. 1995 Mol Microbiol 15:581-589); type VI strain NT6 (Tamura, G. S. & Rubens, C. E. 1995 Mol Microbiol 15:581-589); type VII strain 87-603; type VIII strain JM9 (kindly provided by Pat Ferrieri, University of Minnesota). COH1-13 is an acapsular Tn916ΔE mutant of COH1 (Rubens, C. E. et al. 1993 Mol Microbiol 8:843-855).

Media, Chemicals, and Culture of Bacterial Strains.

E. coli and GBS were grown in Luria Broth and Todd-Hewitt Broth (THB), respectively. Concentrations of antibiotics for selection included: ampicillin (Amp; 75 μg/ml), erythromycin (Erm; 400 μg/ml for E. coli and 10 μg/ml for GBS) or chloramphenicol (Cam; 10 μg/ml). For the culture of GBS in human plasma, plasma was obtained from healthy human donors after consent. GBS was grown to OD₆₀₀ of 0.6 in THB, washed twice in an equal volume of PBS, and resuspended in plasma at a concentration of approximately 1000 CFU/ml. Growth was then monitored over a six hour time period, duplicate dilutions were plated, and doubling time was calculated.

DNA and RNA Methods.

Standard procedures for cloning, sequencing, Southern blotting, Northern blotting, and PCR amplification were utilized (Sambrook, J. E. et al. 1989 Molecular Cloning: a Laboratory Manual Cold Spring Harbor, N.Y. Cold Spring Harbor Laboratory). RNA was isolated by the method of Yim (Yim, H. H. & Rubens, C. E. 1997 Biotechniques 23:229-231). Antisense DIG-labeled probes were utilized for Northern blot procedures as recommended by the manufacturer (Roche Molecular Biochemicals, Indianapolis, Ind., USA).

Identification and Cloning of the cspA Locus.

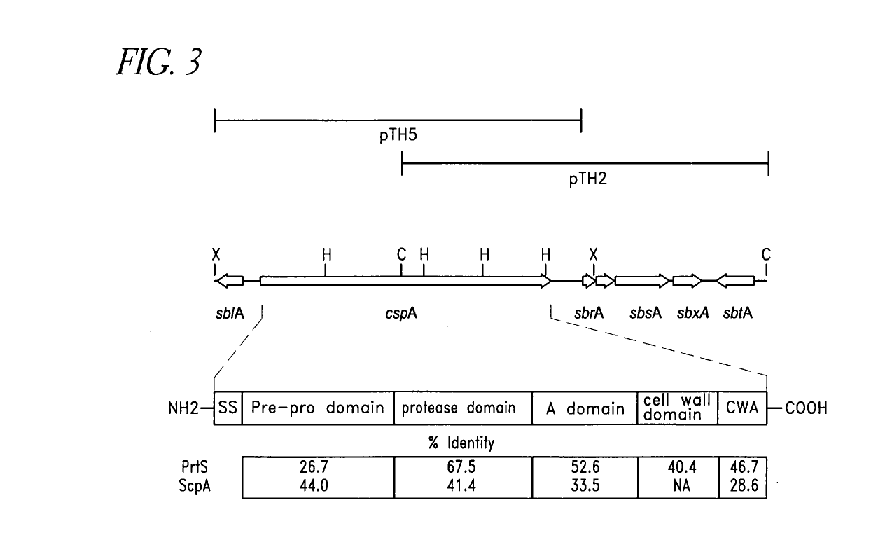

A portion of the cspA open reading frame was originally isolated by analysis of the transposon insertion site from a Tn916AE mutant of strain COH1 and was used as a probe to clone the entire cspA gene. Overlapping ClaI and XbaI restriction fragments (FIG. 3) that bear cspA were identified using Southern analysis of COH1 genomic DNA and cloned into pBSKS- (Stratagene, La Jolla, Calif., USA) using standard techniques. E. coli DH5α clones harboring the desired GBS inserts were identified by colony blots using the probe mentioned above. Clones containing either a ClaI fragment (TOH37 containing plasmid pTH2) or an XbaI fragment (TOH50, containing plasmid pTH5) were further analyzed. The cloned cspA gene present on plasmid pTH5 contains a spontaneous mutation, in comparison to the chromosomal cspA sequence of the wild-type isogenic strain, COH1; this mutation is predicted to terminate translation prematurely at Leu-1121.

Construction of a cspA::erm Mutation in GBS.

To perform allelic replacement mutagenesis of cspA, we subcloned cspA to pVE6007, a broad host range plasmid that replicates at 28° C. but not at 37° C. (Maguin, E. et al. 1992 J Bacteriol 174:5633-5638). A 5.4 kb PCR product of cspA was amplified from COH1 genomic DNA, digested with BamHI and XbaI, and cloned into BamHI/XbaI-digested pVE6007, generating intermediate plasmid pTH19. Approximately 3.6 kb of cspA (corresponding to CspA peptide residues 323-1536) sequence flanked by HindIII sites was subsequently replaced with the erm^{r} gene from pCER1000 (Rubens, C. E. & Heggen, L. M. 1988 Plasmid 20:137-142). The final construct, pTH21 was transformed into COH1 as described (Framson, P. E. et al. 1997 Appl Environ Microbiol 63:3539-3547). A strain that bears a replacement of cspA with the erm^{r} element was obtained by curing the plasmid, as described in (Yim, H. H. & Rubens, C. E. 1998 Methods in Cell Science 20:13-20), and was designated TOH121. The presence of the desired mutation on the chromosome of TOH121 was verified by Southern blotting.

Phenotypic and LD₅₀ Virulence Assays.

Analysis of cell-associated type III GBS capsule (Chaffin, D. O. et al. 2000 J Bacteriol 182:4466-4477), beta-hemolysin, CAMP factor (Nizet, V. et al. 1996 Infect Immun 64:3818-3826), hyaluronidase (Richman, P. G. & Baer, H. 1980 Anal Biochem 109:376-381), and hippuricase expression was performed as described (Ferrieri, P. et al. 1973 Infect Immun 7:747-752). Caseinase activity assayed by growing GBS overnight on THB+5% milk agar plates, in casein zymogram gels (Bio-Rad Laboratories, Hercules, Calif., USA, using GBS culture supernatants or mutanolysin extracts), or by incubating stationary phase whole cell GBS with purified, protease assay grade casein (Sigma, St. Louis, Mo.). Adherence and invasion assays of A549 cell monolayers (type II lung epithelial cells) were performed as described (Winram, S. B. et al. 1998 Methods in Cell Science 20:191-201). C5a protease activity of individual strains was determined by the ability of GBS to inhibit C5a-stimulated adherence of human PMNs to gelatin-coated tissue culture wells as described (Bohnsack, J. F. et al. 1991 Biochem J 273:635-640). Virulence of isogenic cspA^{+/-} strains was compared using a neonatal rat model of lethal GBS infection, by LD₅₀ analysis as described previously (Jones, A. L. et al. 2000 Molecular Microbiology 37:1444-1455); statistical analysis for the LD₅₀ data was performed with the Wilcoxon matched pair signed-ranks test. All animals were maintained according to institutional, state, and federal guidelines.

Recombinant CspA-GST Fusion Protein Production and Generation of CspA Antibody.

A CspA-GST fusion protein was constructed for use as an immunogen. A C-terminal portion of CspA that lacks the putative catalytic domain was amplified from COH1 chromosomal DNA using the following primers, TCGGATCCGCTACTGCTCTAGTT (SEQ ID NO: 3), TTAAGTCGACGTAATGATGCCTTGCTCTA (SEQ ID NO: 4), which incorporate BamHI and SalI sites for cloning. Plasmid pGEX-4T-3 (Amersham-Pharmacia Biosciences, Piscataway, N.J.) was digested with BamHI and SalI and ligated to the PCR product, which was also digested with BamHI and SalI. The CspA-GST fusion protein formed inclusion bodies; after solubilization and SDS-PAGE, CspA was excised from polyacrylamide gels and fragmented as described (Harlow, E. & Lane, D. 1988. Antibodies, a Laboratory Manual Cold Spring Harbor, N.Y. Cold Spring Harbor Laboratory Press p. 68). This preparation was used to immunize a New Zealand white rabbit previously shown to lack antibody (Ab) to the fusion protein.

Western Blot Analysis of CspA.

CspA was released from the periplasmic space of TOH50 (E. coli DH5α harboring plasmid pTH5; see above) by an osmotic shock procedure (Tanaka, T. & Weisblum, B. 1975 J Bacteriol 121:354-362). GBS cell surface associated proteins were extracted by treatment with mutanolysin and subjected to SDS-PAGE (Bohnsack, J. F. et al. 2000 Infect Immun 68:5018-5025). Proteins were transferred to Immobilon-P (Millipore Inc., Bedford, Mass., USA); primary antibody and secondary horseradish peroxidase-conjugated antibody were used at dilutions of 1:500 and 1:1000, respectively. The SuperSignal reagent (Pierce Biotechnology, Inc., Rockford, Ill., USA) was utilized for the chemiluminescent detection of Western blots.

Fibrinogen Degradation Assays.

Purified human fibrinogen, depleted of fibronectin and plasminogen, was purchased from Enzyme Research Laboratories (South Bend, Ind.). To assay fibrinogen degradation, GBS strains COH1 and TOH121 were grown to stationary phase, washed once in phosphate-buffered saline, concentrated 20-fold, and resuspended in PBS. Fibrinogen was then added at a concentration of 0.63 mg/ml. The fibrinogen/cell suspension was incubated with slow rotation at 37° C. After 16 hours, GBS cells were removed by centrifugation. The supernatant was analyzed by SDS-PAGE using large format (18.5×20 cm), 10% acrylamide gels to resolve the species of the fibrinogen alpha chain.

MALDI-TOF MS of Fibrinogen Alpha Species.

The identity of the two alpha chain species was confirmed by MALDI-TOF MS (matrix-assisted laser desorption/ionization time-of-flight mass spectrometry). The fibrinogen alpha chains were separated by SDS-PAGE as described above and the upper and lower-migrating species were excised from the Coomassie-stained gel. The gel slices were destained overnight in 50% methanol. The methanol was removed and acetonitrile was added to cover the gel slices and the mixture was incubated for 10 minutes. The acetonitrile was evaporated under vacuum. Trypsin (sequencing grade, from Promega, Madison, Wis.) was added to the dehydrated gel fragments, incubated for 45 min at 4° C., and then incubated overnight at 37° C. After overnight incubation, the trypsin solution was removed, and the gel slices were extracted twice with 200 μl 5% formic acid, 50% acetonitrile. The trypsin solution was pooled with the extraction solution and evaporated under vacuum. Ten μl of 5% acetonitrile, 0.5% acetic acid was added and 0.6 μl was spotted on the target. Following this, mass spectra were acquired using a BIFLEX III mass spectrometer (Bruker, Billerica, Mass.).

Opsonophagocytosis Assays.

Opsonophagocytosis assays were performed using serum and neutrophils that were obtained after consent from non-immune humans, as previously described (Baltimore, R. S. et al. 1977 J Immunol 118:673-678). All samples were performed in triplicate and controls included samples with heat-inactivated sera (56° C. for 30 min) and without PMN.

Identification of cspA.

It is likely that many factors contribute to the virulence of GBS (Jones, A. L. et al. 2000 Molecular Microbiology 37:1444-1455). In order to identify novel virulence factors, we performed a screen to identify transposon mutants with reduced virulence in GBS. In the process of screening Tn916ΔE mutants of the highly virulent type III GBS isolate, COH1, we identified a gene with homology to cell-surface associated proteases. The ORF, which we designated cspA (FIG. 3), encodes a 1,571 amino acid protein and displays homology to several extracellular serine proteases from the subtilase family. The greatest similarity of CspA (51.2% identity and 58.3% similarity) was to PrtS, which is an extracellular caseinase produced by S. thermophilus. The next highest similarity was to a putative extracellular protease (39% identity and 55% similarity) from the Group A Streptococcus genome sequence (Ferretti, J. J. et al. 2001 PNAS USA 98:4658-4663). The third highest similarity (45% similarity and 36% identity) was to ScpA (Chen, C. C. & Cleary, P. P. 1989 Infect Immun 57:1740-1745) and ScpB (Bohnsack, J. F. et al. 1991 Biochem J 273:635-640), which participate in the proteolytic inactivation of the chemotactic factor C5a in Group A and Group B streptococci, respectively. Additionally, CspA was similar to several caseinases of lactic acid bacteria (36% similarity and 27% identity) encoded by the prtB, prtH, and prtP genes (Siezen, R. J. 1999 Antonie Van Leeuwenhoek 76:139-155).

The sequence of CspA indicates that it shares the functional and structural domains of the cell envelope-associated protease (CEP) family (Siezen, R. J. 1999 Antonie Van Leeuwenhoek 76:139-155). The CEP proteases are a subfamily of subtilisin-like serine proteases that are found in a wide variety of bacteria. An excellent comprehensive review of the properties of the CEP protein family may be found in (Siezen, R. J. 1999 Antonie Van Leeuwenhoek 76:139-155). The motifs required for the catalytic function of this family of serine proteases are present in CspA at residues 168-179 of SEQ ID NO: 2 (aspartic acid motif; VAIIDSGLDTNH), 238-248 of SEQ ID NO: 2 (histidine motif; HGMHVTSIATA) and 565-575 of SEQ ID NO: 2 (serine motif; GTSMASPHVAG); this indicates that CspA functions as a protease.

A general characteristic of caseinases is that they are synthesized as pre-pro enzymes and, following translocation across the cell membrane, are activated by autocatalytic cleavage of a pro-peptide sequence (Siezen, R. J. 1999 Antonie Van Leeuwenhoek 76:139-155). A putative pre-pro domain, spanning residues 1-143, was identified in CspA (Siezen, R. J. 1999 Antonie Van Leeuwenhoek 76:139-155), indicating that CspA can also be synthesized as a pre-pro enzyme and can similarly undergo autocatalytic maturation.

Inactivation of cspA and scpB.

To facilitate the functional analysis of CspA, allelic replacement mutagenesis of cspA was performed. A plasmid bearing a cspA::erm allele was created by deleting a portion of cspA and replacing it with an erythromycin resistance gene (see above). This construct was used to replace the wild-type cspA allele of COH1 (a highly virulent, type III GBS clinical isolate), as detailed above. A single clone, TOH121, was selected for further study and the presence of the cspA::erm mutation on the chromosome of this strain was verified by Southern blotting. To provide controls for evaluating GBS C5a protease activity (see below), a mutant bearing a kanamycin resistance element insertion in the GBS C5a peptidase gene, scpB, was also constructed in the COH1 genetic background, and was designated TOH97. A double mutant bearing both the cspA::erm and the scpB::kan mutations was constructed and designated TOH144. Southern blotting was also used to confirm the presence of the desired mutation for the two scpB::kan mutants.

The cspA Gene is Transcribed as a Monocistronic Operon.

We characterized the transcriptional organization of the region proximal to cspA. Examination of the DNA region next to cspA revealed ORFs located both 5′ and 3′ of cspA (FIG. 3). A gene designated sblA (for Streptococcus group B lipoprotein) is located 5′ of cspA, and is oriented in the opposite transcriptional direction as cspA. The predicted product shares 33% identity with AzlC from Deinococcus radiodurans (White, O. et al. 1999 Science 286:1571-1577); the precise biological function of the AzlC protein is currently unknown (Belitsky, B. R. et al. 1997 J Bacteriol 179:5448-5457). Adjacent to cspA and oriented in the same direction are two putative genes (designated sbrA and sbsA) that encode products with homology to the response regulator and sensor proteins of two-component regulatory systems (Stock, J. B. et al. 1989 Microbiol Rev 53:450-490). A potential promoter sequence is located in the 233 bp of non-coding sequence between cspA and sbrA, suggesting that sbrA and sbsA are transcribed from a promoter that is distinct from that of cspA.

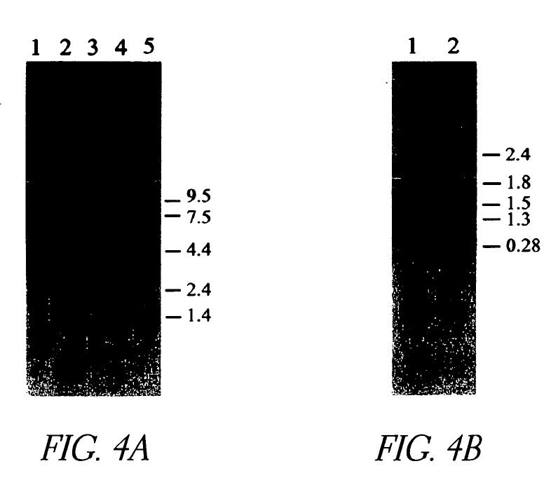

To characterize the transcriptional organization of the cspA region, RNA was isolated from THB-grown COH1 at different growth stages (early-log, mid-log, and stationary phase) and Northern analysis was performed using a probe internal to the cspA coding region (FIG. 4A). A discrete band corresponding to a transcript size of 4.7 kb was present in cells at different stages of growth (FIG. 4A; lanes 1, 2, and 3). A control for probe specificity was performed with strain TOH121; no hybridizing material was seen for this strain, as expected. Maximal expression was observed in early-log phase (O.D.₆₀₀=0.3; lane 1). Since the coding region of cspA is approximately 4.7 kb, these observations suggest that cspA is expressed on a monocistronic transcript.

To confirm our hypothesis that the cspA transcript is monocistronic we performed Northern blot analysis on a gene located 3′ to cspA. It was important to verify that the phenotypes (see below) of the cspA mutant, TOH121, were not due to a polar effect of the cspA mutation onto the adjacent sbrA and sbsA genes (FIG. 3). RNA was isolated from COH1 and TOH121 in mid-log phase and was hybridized to a probe internal to the coding region of sbrA (see FIG. 3). The sbrA-hybridizing material ran as a smear at molecular weights that corresponded to less than 2.4 kb. Similar amounts of hybridizing material were observed in both COH1 and TOH121 (FIG. 4B). The cspA and sbrA probes hybridized to distinct transcripts, suggesting that cspA is transcribed independently from the adjacent genes and that cspA is transcribed as a monocistron. Additionally, similar amounts of each transcript were observed in COH1 and TOH121, confirming that the cspA mutation in TOH121 does not exert a polar effect on expression of the adjacent sbrA gene (FIG. 4B).

CspA is a Surface-Associated, Cell Wall-Anchored Protein.

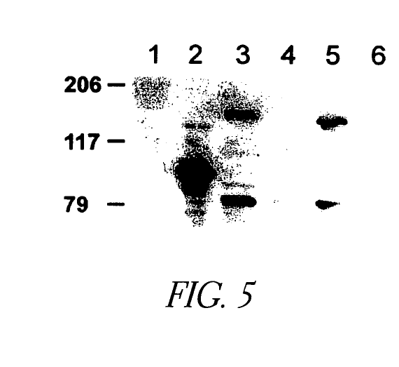

In addition to predicting that CspA functions as a protease, the sequence of CspA suggests that it is secreted and subsequently anchored to the cell surface. A 35 residue signal peptide within the amino terminal end of CspA was identified using SIGSEQ (von Heijne, G. 1986 Nucleic Acid Research 14:4683-4690; FIG. 3). A classical C-terminal cell wall attachment site sequence (LPKTG, amino acids 1536-1540 of SEQ ID NO: 2) characteristic of Gram-positive surface-associated proteins (Navarre, W. W. & Schneewind, O. 1994 Mol Microbiol 14:115-121) was located at amino acids 1536-1540 (FIG. 3 and SEQ ID NO: 2). To investigate the hypothesis that CspA is a surface-attached protein, we determined the subcellular localization of CspA by Western blot analysis. Antibody was raised to a GST-CspA fusion protein expressed from E. coli (see above) and the antibody was used to test different cellular fractions of GBS for the presence of CspA. Periplasmic extracts (see above) of E. coli strain TOH50 (bearing plasmid pTH5; see above) reacted strongly with the antibody raised to GST-CspA. Plasmid pTH5 contains a mutation in the CspA coding region (in comparison to the cspA gene of the wild-type isogenic strain, COH1), that prematurely terminates translation at Leu-1121; this accounts for the lower molecular mass observed for the TOH50 extracts in comparison to wild-type CspA. Proteins from culture supernatants of COH1 did not react with the antibody, even when concentrated ten-fold. In contrast, Western blots of mutanolysin-extracted surface proteins from both COH1 and TOH97 (cspA⁺, scpB⁻) revealed two protein bands of molecular masses 142 and 80 kDa (FIG. 5). The migration of COH1-derived CspA on SDS-PAGE was anomalous, as it corresponded to a lower molecular mass (142 kDa) than predicted by sequence analysis for mature CspA (153 kDa). No cross-reactive bands were seen at the 142 kDa position for the cspA mutants (TOH121 and TOH144), confirming that the antibody does react with wild-type CspA from GBS. The results of the sequence analysis of CspA taken together with the Western blot data indicate the CspA is a surface-localized protein.

CspA does not Function as a C5a Protease.

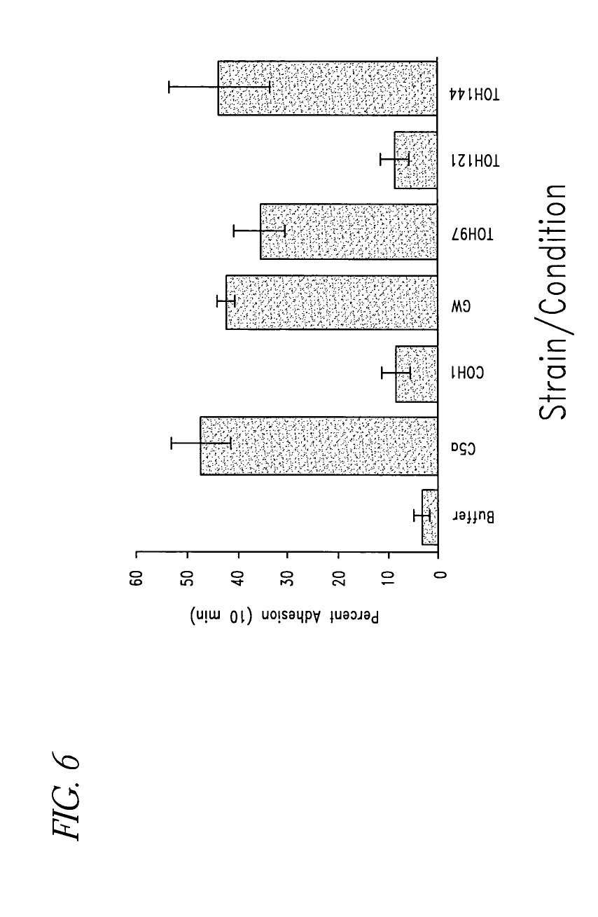

Given the strong similarity of CspA to ScpB, the C5a peptidase of GBS, we hypothesized that CspA may also be active as a C5a protease. We measured C5a-ase activity (FIG. 6) with a functional assay that was described previously (Bohnsack, J. F. et al. 1991 Biochim Biophys Acta 1079:222-228). Briefly, GBS was preincubated with recombinant human C5a, purified human PMN were added, and C5a-stimulated PMN adherence to gelatin-coated plastic was measured. COH1 (wt, scpB+) served as a positive control, and TOH97 (scpB⁻) and another scpB⁻ strain, GW (Bohnsack, J. F. et al. 2000 Infect Immun 68:5018-5025), served as negative controls. The effect of the cspA mutation was measured both in the presence and absence of the GBS C5a protease (ScpB) by comparing TOH121 (scpB⁺, cspA⁻) and TOH144 (scpB⁻, cspA⁻) to the controls. Preincubation of C5a with COH1 or TOH121 abolished C5a activity and the C5a protease activities of the strains were similar (FIG. 6). In contrast, C5a protease activity of strains GW, TOH97, and TOH144 were low and not significantly different from each other. Thus, inactivation of CspA did not appear to have a detectable effect on C5a activity, as evaluated in this functional assay. C5a protease activity only correlated with the presence of functional ScpB. The above results, taken together, are consistent with the conclusion that CspA does not function as a C5a protease.

Evaluation of GBS Phenotypic Traits.

We evaluated the expression of several GBS phenotypic traits, some of which are known GBS virulence determinants. The cspA mutant expressed beta-hemolysin, CAMP factor, hippuricase, and hyaluronidase similarily to wild-type GBS. These strains also expressed equivalent amounts of type III capsule as measured by competitive ELISA (72.7±15.8 for COH1 and 82.9±26.3 μg/mg dry wt for TOH121). Invasion of A549 epithelial cell monolayers was 1.2%±0.3% for COH1 and 1.3%±0.2% for TOH121, expressed as percentage of the total input bacteria invading the A549 cells.

Many of the proteases that are homologous to CspA are important for bacterial growth. For example, caseinases from lactic acid bacteria participate in the degradation of extracellular casein, prior to utilization of the casein peptides as a nutritional source. Because GBS is auxotrophic for multiple amino acids, it must rely on exogenous amino acids. Thus, we compared the growth characteristics of the cpsA mutant to the wild-type strain in different growth media. Growth of the strains was comparable in all media that were tested including RPMI plus 5% casamino acids and THB. Growth in human plasma was compared and the two strains exhibited doubling times of 0.522 hours and 0.598 hours for the COH1 and TOH121, respectively. This indicated that CspA does not play a role in nutritional scavenging, at least under the experimental conditions tested.

Caseinase Activity of GBS.

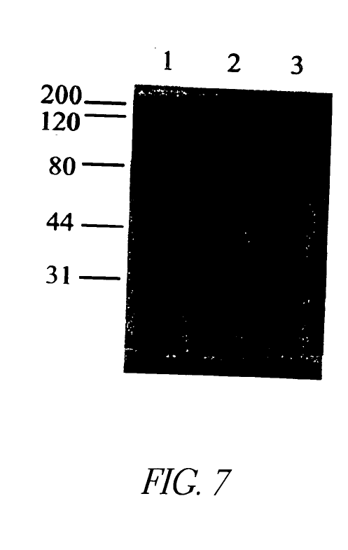

Because of the strong similarity of CspA to PrtS, which functions as a caseinase, we tested whole cells of COH1 and TOH121 for casein degradation. Whole GBS cells were incubated with casein, the mixture was centrifuged, SDS-PAGE was performed on the supernatant, and the amount of intact casein was measured by quantitation of Coomassie-stained gels. Very little casein degradation was observed under the experimental conditions employed, and experiments with the two strains yielded similar results (FIG. 7).

The cspA Gene is Required for Cleavage of Human Fibrinogen.

We hypothesized that CspA, as a putative surface-localized protein, proteolyses a host factor. Therefore, to test the ability of CspA to function as a protease, we compared the ability of the cspA mutant and the wild-type strain to degrade a variety of host proteins. We tested purified human fibronectin, purified complement component C3, and purified human fibrinogen. To evaluate degradation, either whole bacteria and/or mutanolysin-extracted surface proteins of COH1 and TOH121 were incubated with the test substrate and degradation was assessed by evaluating Coomassie-stained SDS-PAGE gels or by Western blotting. No difference in the cleavage of the test substrates was observed for all substrates except fibrinogen when the mutant and wild-type strains were compared.

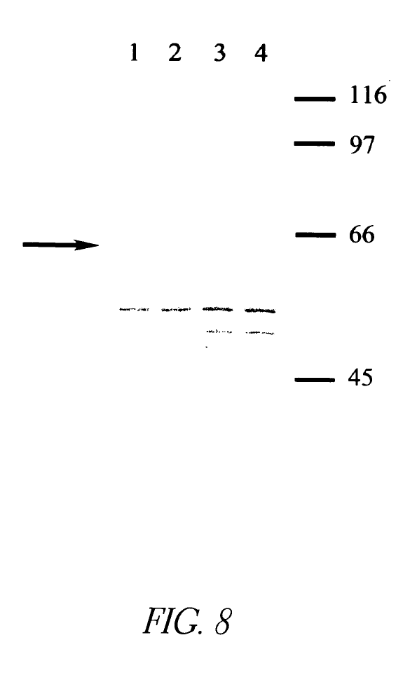

Human fibrinogen was cleaved by the wild-type strain but was not cleaved by the cspA mutant, TOH121. Fibrinogen is a dimer of non-identical subunits, α, β, and γ, that are covalently linked together by disulfide bonds (Doolittle, R. F. 1987 In: Haemostasis and Thrombosis A.L.a.T. Bloom, D. P. ed. London, England, Churchill Livingstone Co.). For fibrinogen isolated from fresh human plasma, we found that SDS-PAGE resolves the alpha subunit of fibrinogen into a doublet. We excised both putative α bands from an SDS-PAGE gel and confirmed their identity by MALDI-TOF mass spectrometry. Peptides matching the sequence of the alpha fragment of human fibrinogen were obtained from both putative alpha bands; 28% of the fibrinogen sequence was covered by the tryptic peptides that were obtained. The lower band of the a doublet is an in vivo cleavage product in which the C-terminal 27 amino acids are removed (Cottrell, B. A. & Doolittle, R. F. 1976 Biochem Biophys Res Commun 71:754-761). When the wild-type and CspA mutants were compared for fibrinogen cleavage, SDS-PAGE revealed that COH1 cleaved the minor species (corresponding to the lower band of the doublet) of the α subunit, while the mutant did not cleave fibrinogen under the conditions employed (FIG. 8). We also noticed that when the mutant and wild-type were incubated with fibrinogen overnight, the wild-type strain formed macroscopic aggregates that were much more prominent in comparison with the mutant strain. These aggregates were not observable in controls in which the wild-type strain was incubated in PBS alone.

CspA is Essential for Virulence in a Neonatal Rat Model.

Extracellular proteases have been implicated in virulence both in gram-negative and gram-positive bacteria. To determine if CspA contributes to virulence, we compared the LD₅₀ of TOH121 (cspA⁻) to the isogenic wild type strain, COH1. A series of ten-fold dilutions of each strain were introduced by intraperitoneal injection into 24-48 hour neonatal rats. Five separate lethality experiments were performed. The mean LD₅₀ values were 2.9×10³ and 2.9.×10⁴ cfu per animal for COH1 and TOH121, respectively (P≦0.0431 by the Wilcoxon matched paired signed-ranks test). These results indicated that mutation of cspA gene significantly impairs the virulence of GBS in the neonatal sepsis model.

CspA Promotes Evasion of Opsonophagocytosis.

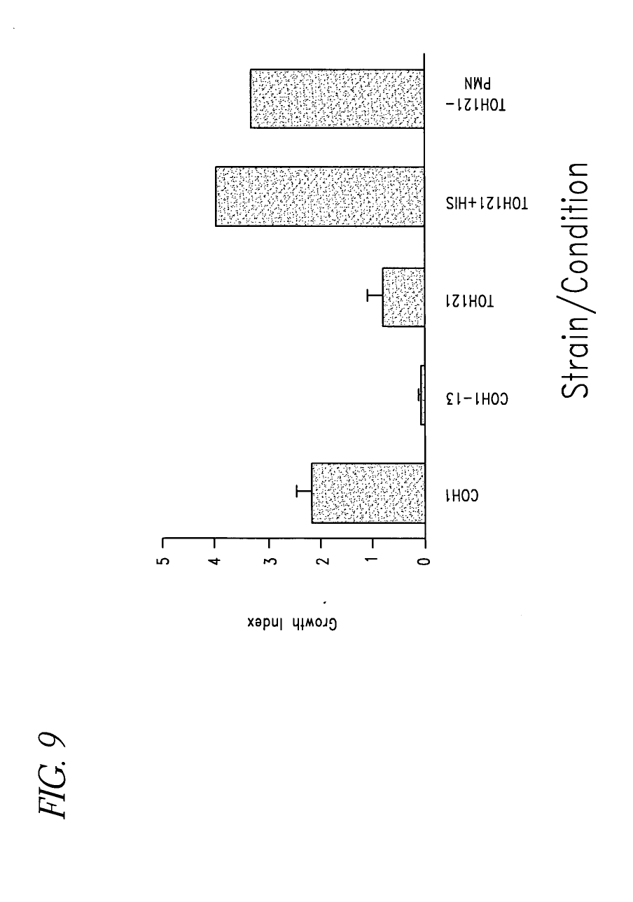

We hypothesized that CspA may allow GBS to avoid innate immune clearance in the non-immune host, perhaps by a novel mechanism, since capsular polysaccharide expression was not affected by the cspA mutation (see above). Opsonophagocytosis is an important mechanism for bacterial clearance, and neutrophils play a major role in the elimination of GBS from the bloodstream (Nizet, V. et al. 2000 In: Streptococcal Infections: Clinical Aspects, Microbiology, and Molecular Pathogenesis New York: Oxford University Press 180-221). We hypothesized that the attenuated virulence of TOH121 may be a consequence of increased susceptibility to phagocytic clearance compared to COH1. Fresh PMN were isolated from human donors and pooled human sera was used as a source of complement and was preabsorbed with COH1 to remove antibodies directed against the bacteria. The assay was repeated 4 times, and the results of a representative experiment are shown in FIG. 7. COH 1-13 (Rubens, C. E. et al. 1993 Mol Microbiol 8:843-855), which is an unencapsulated mutant of COH1 known to be very susceptible to PMN killing, was included as a control. The growth index (GI) of each strain was calculated as the output cfu per ml divided by the input cfu per ml. COH1 growth during the assay corresponded to more than one doubling (GI=2.2; FIG. 9). In contrast, the negative control strain, COH1-13 was markedly killed by human PMN during the 1 h incubation (GI=0.04). TOH121 exhibited a sensitivity that was intermediate between COH1 and COH1-13 (GI=0.81). All three strains grew in the presence of heat-inactivated sera and in the absence of PMNs, yielding growth indices between 2 and 4 (FIG. 9). We also averaged the results of the four experiments and expressed the results as a ratio of the growth index of the mutant to the growth index of the wild type, which was 0.46 (P<0.001 by Student's t test). These findings indicate that CspA promotes resistance to non-immune opsonization and killing by PMNs, though not to the same degree as observed for the capsular polysaccharide (Rubens, C. E. et al. 1993 Mol Microbiol 8:843-855).

The cspA Gene is Widely Distributed Among the GBS Serotypes.

Since CspA contributes to virulence, we investigated the prevalence of cspA in the other GBS serotypes. Southern blots were performed using GBS genomic DNA isolated from representative strains from each of the nine serotypes. A single DNA fragment from 16 of 18 strains hybridized to the cspA probe and DNA from at least one representative of each serotype hybridized to the probe. Therefore, the cspA gene is prevalent among all the serotypes examined, in contrast to most other protective GBS antigens discovered to date.

In summary, we have identified a novel GBS protease with extensive homology to the C5a-ases of GAS and GBS, as well as to other members of the subtilase family of serine proteases. All GBS serotypes that were examined bore the cspA gene. Mutation of cspA attenuated GBS virulence in a neonatal rat sepsis model and decreased resistance to phagocytosis. The protease is necessary for the cleavage human fibrinogen. Combating the contribution of this novel serine protease-like gene to the pathogenesis of GBS disease is contemplated as being the basis for a vaccine to protect against this significant pathogen that causes severe infections of the newborn infant and other immunocompromised individuals.

TABLE 1

| Strain or | | |
|---|---|---|
| Plasmid | Relevant characteristics | Reference or source |
| S. agalactiae | | |
| COH1 | Type III GBS isolate from | (Martin, T.R. et al. 1988 J |
| | neonate with septicemia; | Infect Dis 157: 91-100) |
| | highly virulent; wild-type | |
| | reference strain | |
| TOH121 | cspA:: erm derivative of | This work |
| | COH1; Erm^{R} | |
| TOH97 | scpB:: kan derivative of | (Beckmann, C. et al. 2002 |
| | COH1; Kan^{R} | Infection & Immunity 70) |
| TOH144 | scpB:: kan derivative of | This work |
| | TOH121 (cspA:: erm); Erm^{R}, | |
| | Kan^{R} | |
| E. coli | | |
| DH5α | General cloning strain | ATCC |
| TOH50 | DH5α bearing plasmid pTH5 | This work |
| Plasmids | | |
| PBSKS⁻ | General cloning vector | Stratagene (La Jolla, CA, |
| | | USA) |
| pVE6007 | E. coli-GBS shuttle vector; | (Maguin, E. et al. 1992 J |
| | temperature-sensitive | Bacteriol 174: 5633-5638) |
| | replication; pWV origin of | |
| | replication; Cm^{R} | |
| pTH2 | pBSKS bearing ClaI DNA | This work |
| | fragment of cspA; Amp^{R} | |
| pTH5 | pBSKS bearing XbaI DNA | This work |
| | fragment containing cspA; | |
| | Amp^{R} | |
| pTH21 | Plasmid used for insertional | This work |
| | mutagenesis of cspA; | |
| | Derivative of pVE6007; | |
| | Cm^{R}; Erm^{R} | |

### Use of a Novel Cell Surface Protease Protein of Group B Streptococcus (GBS), called CspA, as a Vaccine to Prevent GBS Infection

According to one aspect of the invention, we provide a CspA protease of Group B Streptococcus, and analogues, homologues, derivatives and fragments thereof, containing at least one immunogenic epitope. As used herein, a “CspA protease” is a naturally occurring protein that has amino acid sequence of SEQ ID NO: 2. The CspA protease according to the invention may be of natural origin, or may be obtained through the application of recombinant DNA techniques, or conventional chemical synthesis techniques.

As used herein, “immunogenic” means having the ability to elicit an immune response. The novel CspA protease of this invention is characterized by its ability to elicit a protective immune response against Group B Streptococcus infection.

The invention particularly provides a CspA protease of Group B Streptococcus of approximately 172 kDa, having the deduced amino acid sequence of SEQ ID NO: 2, and analogues, homologues, derivatives and fragments thereof, containing at least one immunogenic epitope.

As used herein, “analogues” of CspA protease are those Group B Streptococcus proteins wherein one or more amino acid residues in the CspA protease amino acid sequence (SEQ ID NO: 2) is replaced by another amino acid residue, providing that the overall functionality and immunogenic properties of the analogue protein are preserved. Such analogues may be naturally occurring, or may be produced synthetically or by recombinant DNA technology, for example, by mutagenesis of the cspA protease sequence. Analogues of CspA protease will possess at least one antigen capable of eliciting antibodies that react with CspA protease. Such an analogue can have overall homology (i.e., similarity) or identity of at least 80% to CspA protein, such as 80-99% homology (i.e., similarity) or identity, or any range therein.

Percent homology can be determined, for example, by comparing sequence information using the GAP computer program, version 6.0, available from the University of Wisconsin Genetics Computer Group (UWGCG). The GAP program utilizes the alignment method of Needleman and Wunsch (J Mol Biol 1970 48:443), as revised by Smith and Waterman (Adv Appl Math 1981 2:482). Briefly, the GAP program defines similarity as the number of aligned symbols (i.e., nucleotides or amino acids) which are similar, divided by the total number of symbols in the shorter of the two sequences. The preferred default parameters for the GAP program include: (1) a unitary comparison matrix (containing a value of 1 for identities and 0 for non-identities) and the weighted comparison matrix of Gribskov and Burgess (Nucl Acids Res 1986 14:6745), as described by Schwartz and Dayhoff, eds. (Atlas of Protein Sequence and Structure, National Biomedical Research Foundation, Washington, D.C. 1979, pp. 353-358); (2) a penalty of 3.0 for each gap and an additional 0.10 penalty for each symbol in each gap; and (3) no penalty for end gaps.

As used herein, “homologues” of CspA protease are proteins from Streptococcal species other than agalactiae, or genera other than Streptococcus wherein one or more amino acid residues in the CspA protease amino acid sequence (SEQ ID NO: 2) is replaced by another amino acid residue, providing that the overall functionality and immunogenic properties of the homologue protein are preserved. Such homologues may be naturally occurring, or may be produced synthetically or by recombinant DNA technology. Homologues of CspA protease will possess at least one antigen capable of eliciting antibodies that react with CspA protease. Such a homologue can have overall homology (i.e., similarity) or identity of at least 80% to CspA protein, such as 80-99% homology (i.e., similarity) or identity, or any range therein.

Again, percent homology can be determined, for example, by comparing sequence information using the GAP computer program, version 6.0, available from the University of Wisconsin Genetics Computer Group (UWGCG). The GAP program utilizes the alignment method of Needleman and Wunsch (J Mol Biol 1970 48:443), as revised by Smith and Waterman (Adv Appl Math 1981 2:482). Briefly, the GAP program defines similarity as the number of aligned symbols (i.e., nucleotides or amino acids) which are similar, divided by the total number of symbols in the shorter of the two sequences. The preferred default parameters for the GAP program include: (1) a unitary comparison matrix (containing a value of 1 for identities and 0 for non-identities) and the weighted comparison matrix of Gribskov and Burgess (Nucl Acids Res 1986 14:6745), as described by Schwartz and Dayhoff, eds. (Atlas of Protein Sequence and Structure, National Biomedical Research Foundation, Washington, D.C. 1979, pp. 353-358); (2) a penalty of 3.0 for each gap and an additional 0.10 penalty for each symbol in each gap; and (3) no penalty for end gaps.

As used herein, a “derivative” is a polypeptide in which one or more physical, chemical, or biological properties has been altered. Such alterations include, but are not limited to: amino acid substitutions, modifications, additions or deletions; alterations in the pattern of lipidation, glycosylation or phosphorylation; reactions of free amino, carboxyl, or hydroxyl side groups of the amino acid residues present in the polypeptide with other organic and non-organic molecules; and other alterations, any of which may result in changes in primary, secondary or tertiary structure.

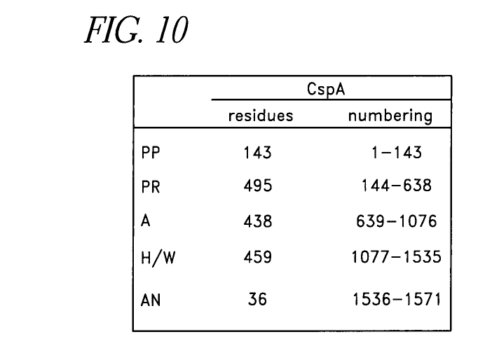

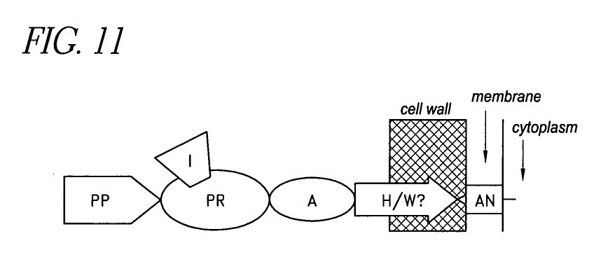

The “fragments” of this invention will have at least one immunogenic epitope. An “immunogenic epitope” is an epitope that is instrumental in eliciting an immune response. The preferred fragments of this invention will elicit an immune response sufficient to prevent or ameliorate the severity of infection. Referring to FIG. 10 and FIG. 11, the multi-domain, cell-envelope proteinase encoded by the gene cspA of Streptococcus agalactiae has been compared to other sequences using multiple sequence alignment, secondary structure prediction and database homology searching methods. This comparative analysis had led to the prediction of a number of different domains in this cell-envelope proteinase. These domains include, starting from the N-terminus, a pre-pro-domain for secretion and activation, a serine protease domain (with a smaller inserted domain), a large middle domain A of unknown but possibly regulatory function, a helical spacer domain, a hydrophilic cell-wall spacer or attachment domain, and a cell-wall anchor domain. Preferred fragments of CspA protease include pre-pro domain (residues 1-143), protease domain (144-638), A domain (639-1076), cell-wall spacer domain (1077-1535), and cell-wall anchor domain (1536-1571), and smaller fragments consisting of peptide epitopes within any of the above mentioned domains.

Removing a major portion of the amino-terminal end of the protein to make the GST-CspA fusion described above was designed to eliminate the protease activity while maximizing epitope presentation. The catalytic domain for the CspA serine protease is most likely located between aa 168-575, and contains the three motifs common to other well known serine proteases (see description above). Theoretically, a mutation in one or more of the three motifs or potentially elsewhere within this region could either abolish protease function or alter the conformation of the active catalytic site(s) effecting protease activity. Alternatively, since like other serine proteases, these enzymes also contain “pre-pro” domains from aa 1-143, mutations preventing maturation of the protein to its “active” form may eliminate protease activity. Substitution mutations or larger deletions of the protein are also possibilities. As described in the examples, antibody raised to the GST-CspA fusion protein was protective. Therefore, this data indicate that removing a major portion of the amino-terminal end of the protein and presumably eliminating protease activity has a limited impact on antigenicity.

In a further aspect of the invention, we provide polypeptides that are immunologically related to CspA protease. As used herein, “immunologically related” polypeptides are characterized by one or more of the following properties:
(a) they are immunologically reactive with antibodies generated by infection of a mammalian host with Group B Streptococcus cells, which antibodies are immunologically reactive with CspA protease;
(b) they are capable of eliciting antibodies that are immunologically reactive with CspA protease;
(c) they are immunologically reactive with antibodies elicited by immunization of a mammal with CspA protease.

By definition, analogues, homologues and derivatives of CspA protease are immunologically related polypeptides.

Moreover, all immunologically related polypeptides contain at least one CspA protease antigen. Accordingly, “CspA protease antigens” may be found in CspA protease itself, or in immunologically related polypeptides.

As used herein, “related bacteria” are bacteria that possess antigens capable of eliciting antibodies that react with CspA protease. Examples of related bacteria include Group A Streptococcus.

It will be understood that one of skill in the art may determine without undue experimentation whether a particular analogue, homologue, derivative, immunologically related polypeptide, or fragment would be useful in the prevention or treatment of disease. Useful polypeptides and fragments will elicit antibodies that are immunoreactive with CspA protease. Preferably, useful polypeptides and fragments will demonstrate the ability to elicit a protective immune response against lethal bacterial infection.

Also included are polymeric forms of the polypeptides of this invention. These polymeric forms include, for example, one or more polypeptides that have been crosslinked with crosslinkers such as avidin/biotin, glutaraldehyde or dimethylsuberimidate. Such polymeric forms also include polypeptides containing two or more tandem or inverted contiguous protein sequences, produced from multicistronic mRNAs generated by recombinant DNA technology.

This invention provides substantially pure CspA protease and immunologically related polypeptides. The term “substantially pure” means that the polypeptides according to the invention, and the DNA sequences encoding them, are substantially free from other proteins of bacterial origin. Substantially pure protein preparations may be obtained by a variety of conventional processes.

In another aspect, this invention provides, for the first time, a DNA sequence coding for a CspA protease of group B Streptococcus having SEQ ID NO: 1.

The DNA sequences of this invention also include DNA sequences coding for polypeptide analogues and homologues of CspA protease, DNA sequences coding for immunologically related polypeptides, DNA sequences that are degenerate to any of the foregoing DNA sequences, and fragments of any of the foregoing DNA sequences. It will be readily appreciated that a person of ordinary skill in the art will be able to determine the DNA sequence of any of the polypeptides of this invention, once the polypeptide has been identified and isolated, using conventional DNA sequencing techniques.

The polypeptides of this invention may be prepared from a variety of processes, for example by protein fractionization from appropriate cell extracts, using conventional separation techniques such as ion exchange and gel chromatography and electrophoresis, or by the use of recombinant DNA techniques. The use of recombinant DNA techniques is particularly suitable for preparing substantially pure polypeptides according to the invention.

Thus according to a further aspect of the invention, we provide a process for the production of CspA protease, immunologically related polypeptides, and fragments thereof, comprising the steps of (1) culturing a unicellular host organism transformed with a vector containing a DNA sequence coding for said polypeptide or fragment thereof and one or more expression control sequences operatively linked to the DNA sequence, and (2) recovering a substantially pure polypeptide or fragment.

As is well known in the art, in order to obtain high expression levels of a transfected gene in a host, the gene must be operatively linked to transcriptional and translational expression control sequences that are functional in the chosen expression host. Preferably, the expression control sequences, and the gene of interest, will be contained in an expression vector that further comprises a bacterial selection marker and origin of replication. If the expression host is an eukaryotic cell, the expression vector should further comprise an expression marker useful in the eukaryotic expression host.

The DNA sequences encoding the polypeptides of this invention may or may not encode a signal sequence. If the expression host is eukaryotic, it generally is preferred that a signal sequence be encoded so that the mature protein is secreted from the eukaryotic host.

An amino terminal methionine may or may not be present on the expressed polypeptides of this invention. If the terminal methionine is not cleaved by the expression host, it may, if desired, be chemically removed by standard techniques.

A wide variety of expression host/vector combinations may be employed in expressing the DNA sequences of this invention. Useful expression vectors for eukaryotic hosts include, for example, vectors comprising expression control sequences from SV40, bovine papilloma virus, adenovirus, adeno-associated virus, cytomegalovirus, and retroviruses. Useful expression vectors for bacterial hosts include bacterial plasmids, such as those from E. coli, including pbluescript, pGEX2T, pUC vectors, col E1, pCR1, pBR322, pMB9 and their derivatives, wider host range plasmids, such as RP4, phage DNAs, e.g., the numerous derivatives of phage λ, e.g., λGT10 and λGT11, NM989, and other DNA phages, such as M13 and filamentous single stranded DNA phages. Useful expression vectors for yeast cells include the 2μ plasmid and derivatives thereof. Useful vectors for insect cells include pVL 941.

In addition, any of a wide variety of expression control sequences may be used in these vectors to express the DNA sequences of this invention. Useful expression control sequences include the expression control sequences associated with structural genes of the foregoing expression vectors. Examples of useful expression control sequences include, for example, the early and late promoters of SV40 or adenovirus, the lac system, the trp system, the TAC or TRC system, the T3 and T7 promoters the major operator and promoter regions of phage lambda, the control regions of fd coat protein, the promoter for 3-phosphoglycerate kinase or other glycolytic enzymes, the promoters of acid phosphatase, e.g., Pho5, the promoters of the yeast alpha-mating system and other constitutive and inducible promoter sequences known to control the expression of genes of prokaryotic or eukaryotic cells or their viruses, and various combinations thereof. The T7 RNA polymerase promoter Φ10 is particularly useful in the expression of CspA protease in E. coli.

Host cells transformed with the foregoing vectors form a further aspect of this invention. A wide variety of unicellular host cells are useful in expressing the DNA sequences of this invention. These hosts may include well known eukaryotic and prokaryotic hosts, such as strains of E. coli, Pseudomonas, Bacillus, Streptomyces, fungi, yeast, insect cells such as Spodoptera frugiperda (SF9), animal cells such as CHO and mouse cells, African green monkey cells such as COS 1, COS 7, BSC 1, BSC 40, and BMT 10, human cells, and plant cells in tissue culture. Preferred host organisms include bacteria such as E. coli and B. subtilis, and mammalian cells in tissue culture.

It should, of course, be understood that not all vectors and expression control sequences will function equally well to express the DNA sequences of this invention. Neither will all hosts function equally well with the same expression system. However, one of skill in the art may make a selection among these vectors, expression control sequences and hosts without undue experimentation and without departing from the scope of this invention. For example, in selecting a vector, the host must be considered because the vector must replicate in it. The vector's copy number, the ability to control that copy number, and the expression of any other proteins encoded by the vector, such as antibiotic markers, should also be considered. In selecting an expression control sequence, a variety of factors should also be considered. These include, for example, the relative strength of the sequence, its controllability, and its compatibility with the DNA sequences of this invention, particularly as regards potential secondary structures. Unicellular hosts should be selected by consideration of their compatibility with the chosen vector, the toxicity of the product coded for by the DNA sequences of this invention, their secretion characteristics, their ability to fold the protein correctly, their fermentation or culture requirements, and the ease of purification from them of the products coded for by the DNA sequences of this invention. Within these parameters, one of skill in the art may select various vector/expression control sequence/host combinations that will express the DNA sequences of this invention on fermentation or in large-scale animal culture.

The polypeptides encoded by the DNA sequences of this invention may be isolated from the fermentation or cell culture and purified using any of a variety of conventional methods including: liquid chromatography such as normal or reversed phase, using HPLC, FPLC and the like; affinity chromatography (such as with inorganic ligands or monoclonal antibodies); size exclusion chromatography; immobilized metal chelate chromatography; gel electrophoresis; and the like. One of skill in the art may select the most appropriate isolation and purification techniques without departing from the scope of this invention.

In addition, the polypeptides of this invention may be generated by any of several chemical techniques. For example, they may be prepared using the solid-phase synthetic technique originally described by R. B. Merrifield (J Am Chem Soc 1963 83:2149-54), or they may be prepared by synthesis in solution. A summary of peptide synthesis techniques may be found in E. Gross & H. J. Meinhofer, 4 The Peptides: Analysis Synthesis, Biology; Modern Techniques Of Peptide And Amino Acid Analysis, John Wiley & Sons, (1981); and M. Bodanszky, Principles Of Peptide Synthesis, Springer-Verlag (1984).

The preferred compositions and methods of this invention comprise polypeptides having enhanced immunogenicity. Such polypeptides may result when the native forms of the polypeptides or fragments thereof are modified or subjected to treatments to enhance their immunogenic character in the intended recipient. Numerous techniques are available and well known to those of skill in the art which may be used, without undue experimentation, to substantially increase the immunogenicity of the polypeptides herein disclosed. For example, the polypeptides may be modified by coupling to dinitrophenol groups or arsanilic acid, or by denaturation with heat and/or SDS. Particularly if the polypeptides are small polypeptides synthesized chemically, it may be desirable to couple them to an immunogenic carrier. The coupling of course, must not interfere with the ability of either the polypeptide or the carrier to function appropriately. For a review of some general considerations in coupling strategies, see Antibodies, A Laboratory Manual, Cold Spring Harbor Laboratory, ed. E. Harlow and D. Lane (1988). Useful immunogenic carriers are well known in the art. Examples of such carriers are keyhole limpet hemocyanin (KLH); albumins such as bovine serum albumin (BSA) and ovalbumin, PPD (purified protein derivative of tuberculin); red blood cells; tetanus toxoid; cholera toxoid; agarose beads; activated carbon; or bentonite.

Modification of the amino acid sequence of the polypeptides disclosed herein in order to alter the lipidation state is also a method which may be used to increase their immunogenicity and biochemical properties. For example, the polypeptides or fragments thereof may be expressed with or without the signal sequences that direct addition of lipid moieties.

In accordance with this invention, derivatives of the polypeptides may be prepared by a variety of methods, including by in vitro manipulation of the DNA encoding the native polypeptides and subsequent expression of the modified DNA, by chemical synthesis of derivatized DNA sequences, or by chemical or biological manipulation of expressed amino acid sequences.

For example, derivatives may be produced by substitution of one or more amino acids with a different natural amino acid, an amino acid derivative or non-native amino acid, conservative substitution being preferred, e.g., 3-methylhistidine may be substituted for histidine, 4-hydroxyproline may be substituted for proline, 5-hydroxylysine may be substituted for lysine, and the like.

Causing amino acid substitutions which are less conservative may also result in desired derivatives, e.g., by causing changes in charge, conformation and other biological properties. Such substitutions would include for example, substitution of a hydrophilic residue for a hydrophobic residue, substitution of a cysteine or proline for another residue, substitution of a residue having a small side chain for a residue having a bulky side chain, or substitution of a residue having a net positive charge for a residue having a net negative charge. When the result of a given substitution cannot be predicted with certainty, the derivatives may be readily assayed according to the methods disclosed herein to determine the presence or absence of the desired characteristics.

The polypeptides may also be prepared with the objective of increasing stability, facilitating purification, or making a multimeric vaccine. One such technique is to express the polypeptides as fusion proteins comprising other Group B Streptococcus or non-Group B Streptococcus sequences. It is preferred that the fusion proteins comprising the polypeptides of this invention be produced at the DNA level, e.g., by constructing a nucleic acid molecule encoding the fusion protein, transforming host cells with the molecule, inducing the cells to express the fusion protein, and recovering the fusion protein from the cell culture. Alternatively, the fusion proteins may be produced after gene expression according to known methods.

The polypeptides of this invention may also be part of larger multimeric molecules which may be produced recombinantly or may be synthesized chemically. Such multimers may also include the polypeptides fused or coupled to moieties other than amino acids, including lipids and carbohydrates.

The polypeptides of this invention are particularly well-suited for the generation of antibodies and for the development of a protective response against disease. Accordingly, in another aspect of this invention, we provide antibodies, or fragments thereof, that are immunologically reactive with CspA protease. The antibodies of this invention are either elicited by immunization with CspA protease or an immunologically related polypeptide, or are identified by their reactivity with CspA protease or an immunologically related polypeptide. It should be understood that the antibodies of this invention are not intended to include those antibodies which are normally elicited in an animal upon infection with naturally occurring Group B Streptococcus and which have not been removed from or altered within the animal in which they were elicited.

The antibodies of this invention may be intact immunoglobulin molecules or fragments thereof that contain an intact antigen binding site, including those fragments known in the art as F(v), Fab, Fab′ and F(ab′)₂. The antibodies may also be genetically engineered or synthetically produced. The antibody or fragment may be of animal origin, specifically of mammalian origin, and more specifically of murine, rat or human origin. It may be a natural antibody or fragment, or if desired, a recombinant antibody or fragment. The antibody or antibody fragments may be of polyclonal, or preferably, of monoclonal origin. They may be specific for a number of epitopes but are preferably specific for one. One of skill in the art may use the polypeptides of this invention to produce other monoclonal antibodies which could be screened for their ability to confer protection against Group B Streptococcus or Group B Streptococcus-related bacterial infection when used to immunize naive animals. Once a given monoclonal antibody is found to confer protection, the particular epitope that is recognized by that antibody may then be identified. Methods to produce polyclonal and monoclonal antibodies are well known to those of skill in the art. For a review of such methods, see Antibodies, A Laboratory Manual, Cold Spring Harbor Laboratory, ed. E. Harlow and D. Lane (1988), and D. E. Yelton et al. 1981 Ann Rev Biochem 50:657-80. Determination of immunoreactivity with a polypeptide of this invention may be made by any of several methods well known in the art, including by immunoblot assay and ELISA.

An antibody of this invention may also be a hybrid molecule formed from immunoglobulin sequences from different species (e.g., mouse and human) or from portions of immunoglobulin light and heavy chain sequences from the same species. It may be a molecule that has multiple binding specificities, such as a bifunctional antibody prepared by any one of a number of techniques known to those of skill in the art including: the production of hybrid hybridomas; disulfide exchange; chemical cross-linking; addition of peptide linkers between two monoclonal antibodies; the introduction of two sets of immunoglobulin heavy and light chains into a particular cell line; and so forth.

The antibodies of this invention may also be human monoclonal antibodies, for example those produced by immortalized human cells, by SCID-hu mice or other non-human animals capable of producing “human” antibodies, or by the expression of cloned human immunoglobulin genes.

In sum, one of skill in the art, provided with the teachings of this invention, has available a variety of methods which may be used to alter the biological properties of the antibodies of this invention including methods which would increase or decrease the stability or half-life, immunogenicity, toxicity, affinity or yield of a given antibody molecule, or to alter it in any other way that may render it more suitable for a particular application.

The polypeptides, DNA sequences and antibodies of this invention are useful in prophylactic, therapeutic and diagnostic compositions for preventing, treating and diagnosing disease.

Standard immunological techniques may be employed with the polypeptides and antibodies of this invention in order to use them as immunogens and as vaccines. In particular, any suitable host may be injected with a pharmaceutically effective amount of polypeptide to generate monoclonal or polyvalent antibodies or to induce the development of a protective immunological response against disease.

As used herein, a “pharmaceutically effective amount” of a polypeptide or of an antibody is the amount that, when administered to a patient, elicits an immune response that is effective to prevent or ameliorate the severity of Group B Streptococcus or related bacterial infections.

The administration of the polypeptides or antibodies of this invention may be accomplished by any standard procedures. For a detailed discussion of such techniques, see Antibodies, A Laboratory Manual, Cold Spring Harbor Laboratory, ed. E. Harlow and D. Lane (1988). Preferably, if a polypeptide is used, it will be administered with a pharmaceutically acceptable adjuvant, such as complete or incomplete Freund's adjuvant, RIBI (muramyl dipeptides) or ISCOM (immunostimulating complexes). For example, the composition will include a water-in-oil emulsion or aluminum hydroxide as adjuvant and will be administered intramuscularly. The vaccine composition may be administered to the patient at one time or over a series of treatments. The most effective mode of administration and dosage regimen will depend upon the level of immunogenicity, the particular composition and/or adjuvant used for treatment, the severity and course of the expected infection, previous therapy, the patient's health status and response to immunization, and the judgment of the treating physician. For example, in an immunocompetent patient, the more highly immunogenic the polypeptide, the lower the dosage and necessary number of immunizations. Similarly, the dosage and necessary treatment time will be lowered if the polypeptide is administered with an adjuvant.

Generally, the dosage will consist of an initial injection, most probably with adjuvant, of about 0.01 to 10 mg, and preferable 0.1 to 1.0 mg, CspA antigen per patient, followed most probably by one or maybe more booster injections. Preferably, boosters will be administered at about 1 and 6 months after the initial injection.

Any of the polypeptides of this invention may be used in the form of a pharmaceutically acceptable salt. Suitable acids and bases which are capable of forming salts with the polypeptides of the present invention are well known to those of skill in the art, and include inorganic and organic acids and bases.

To screen the polypeptides and antibodies of this invention for their ability to confer protection against diseases caused by Group B Streptococcus or related bacteria, or their ability to ameliorate the severity of such infection, one of skill in the art will recognize that a number of animal models may be used. Any animal that is susceptible to infection with Group B Streptococcus or related bacteria may be useful. For example, Balb/c mice are an animal model for active immunoprotection screening, and severe-combined immunodeficient mice are an animal model for passive screening. Thus, by administering a particular polypeptide or antibody to these animal models, one of skill in the art may determine without undue experimentation whether that polypeptide or antibody would be useful in the methods and compositions claimed herein.

According to another embodiment of this invention, we describe a method which comprises the steps of treating a patient with a vaccine comprising a pharmaceutically effective amount of any of the polypeptides of this invention in a manner sufficient to prevent or ameliorate the severity, for some period of time, of Group B Streptococcus or related bacterial infection.

The invention still further provides “genetic immunization” in which genes encoding such immunogens or epitopes of interest from recombinant vectors are administered through immunization using appropriately engineered mammalian expression systems including but not limited to poxviruses, herpesviruses, adenoviruses, alphavirus-based strategies, and naked or formulated DNA-based immunogens. Techniques for engineering such recombinant vectors are known in the art. With respect to techniques for these immunization vehicles and state-of-the-art knowledge, mention is particularly made of: Hormaeche and Kahn, Perkus and Paoletti, Shiver et al., all in: Concepts in Vaccine Development, Kaufman, S. H. E., ed., Walter deGruytes, New York, 1996, and vectors described in Viruses in Human Gene Therapy, Vos, J.-M. H., ed., Chapman and Hall, Carolina Academic Press, New York, 1995, and in Recombinant Vectors in Vaccine Development, Brown, F., ed., Karger, New York, 1994.

The polypeptides, DNA sequences and antibodies of this invention may also form the basis for diagnostic methods and kits for the detection of pathogenic organisms. Several diagnostic methods are possible. For example, this invention provides a method for the detection of Group B Streptococcus or related bacteria in a biological sample comprising the steps of:
(a) isolating the biological sample from a patient;
(b) incubating an antibody of this invention, or fragment thereof with the biological sample to form a mixture; and
(c) detecting specifically bound antibody or fragment in the mixture which indicates the presence of Group B Streptococcus or related bacteria.

Alternatively, this invention provides a method for the detection of antibodies specific to Group B Streptococcus or related bacteria in a biological sample comprising:
(a) isolating the biological sample from a patient;
(b) incubating a polypeptide of this invention or fragment thereof, with the biological sample to form a mixture; and
(c) detecting specifically bound polypeptide in the mixture which indicates the presence of antibodies specific to Group B Streptococcus or related bacteria.

One of skill in the art will recognize that these diagnostic tests may take several forms, including an enzyme-linked immunosorbent assay (ELISA), a radioimmunoassay or a latex agglutination assay.

The diagnostic agents may be included in a kit which may also comprise instructions for use and other appropriate reagents, preferably a means for detecting when the polypeptide or antibody is bound. For example, the polypeptide or antibody may be labeled with a detection means that allows for the detection of the polypeptide when it is bound to an antibody, or for the detection of the antibody when it is bound to Group B Streptococcus or related bacteria. The detection means may be a fluorescent labeling agent such as fluorescein isocyanate (FIC), fluorescein isothiocyanate (FITC), and the like, an enzyme, such as horseradish peroxidase (HRP), glucose oxidase or the like, a radioactive element such as ¹²⁵I or ⁵¹Cr that produces gamma ray emissions, or a radioactive element that emits positrons which produce gamma rays upon encounters with electrons present in the test solution, such as ¹¹C, ¹⁵O, or ¹³N. Binding may also be detected by other methods, for example via avidin-biotin complexes. The linking of the detection means is well known in the art. For instance, monoclonal antibody molecules produced by a hybridoma may be metabolically labeled by incorporation of radioisotope-containing amino acids in the culture medium, or polypeptides may be conjugated or coupled to a detection means through activated functional groups.

The DNA sequences of this invention may be used to design DNA probes for use in detecting the presence of Group B Streptococcus or related bacteria in a biological sample. The probe-based detection method of this invention comprises the steps of:
(a) isolating the biological sample from a patient;
(b) incubating a DNA probe having a DNA sequence of this invention with the biological sample to form a mixture; and
(c) detecting specifically bound DNA probe in the mixture which indicates the presence of Group B Streptococcus or related bacteria.

The DNA probes of this invention may also be used for detecting circulating nucleic acids in a sample, for example using a polymerase chain reaction, as a method of diagnosing Group B Streptococcus or related bacterial infections. The probes may be synthesized using conventional techniques and may be immobilized on a solid phase, or may be labeled with a detectable label. A preferred DNA probe for this application is an oligomer having a sequence complementary to at least about 6 contiguous nucleotides of cspA (SEQ ID NO: 1).

The polypeptides of this invention may also be used to purify antibodies directed against epitopes present on the protein, for example, using immunoaffinity purification of antibodies on an antigen column.

The antibodies or antibody fragments of this invention may be used to prepare substantially pure proteins according to the invention, for example, using immunoaffinity purification of antibodies on an antigen column.

To formulate a vaccine for human use, appropriate CspA antigens may be selected from the polypeptides described herein. For example, one of skill in the art could design a vaccine around the CspA polypeptide or fragments thereof containing an immunogenic epitope. The use of molecular biology techniques is particularly well-suited for the preparation of substantially pure recombinant antigens.

The vaccine composition may take a variety of forms. These include, for example solid, semi-solid and liquid dosage forms, such as powders, liquid solutions or suspensions, and liposomes. Based on our teaching that the CspA antigens of this invention elicit a protective immune response when administered to a human, the compositions of this invention will be similar to those used for immunizing humans with other proteins and polypeptides, e.g., tetanus and diphtheria. Therefore, the compositions of this invention will preferably comprise a pharmaceutically acceptable adjuvant such as incomplete Freund's adjuvant, aluminum hydroxide, a muramyl peptide, a water-in oil emulsion, a liposome, an ISCOM or CTB, or a non-toxic B subunit from cholera toxin. Most preferably, the compositions will include a water-in-oil emulsion or aluminum hydroxide as adjuvant.

The composition would be administered to the patient in any of a number of pharmaceutically acceptable forms including intramuscular, intradermal, subcutaneous, vaginal or topical. Preferably, the vaccine will be administered intramuscularly.

Generally, the dosage will consist of an initial injection, most probably with adjuvant, of about 0.01 to 10 mg, and preferably 0.1 to 1.0 mg CspA antigen per patient, followed most probably by one or more booster injections. Preferably, boosters will be administered at about 1 and 6 months after the initial injection.

An important consideration relating to Streptococcal vaccine development is the question of mucosal immunity. The ideal mucosal vaccine will be safely taken orally or intranasally as one or a few doses and would elicit protective antibodies on the appropriate surfaces along with systemic immunity. The mucosal vaccine composition may include adjuvants, inert particulate carriers or recombinant live vectors.

The anti-CspA antibodies of this invention are useful for passive immunotherapy and immunoprophylaxis of humans infected with Group B Streptococcus or related bacteria. The dosage forms and regimens for such passive immunization would be similar to those of other passive immunotherapies.

The preparation of vaccines based on attenuated microorganisms is known to those skilled in the art. Vaccine compositions can be formulated with suitable carriers or adjuvants, e.g., alum, as necessary or desired, and used in therapy, to provide effective immunization against Group B Streptococci or other related microorganisms. The preparation of vaccine formulations will be apparent to the skilled person.

In another aspect, the present invention relates to a method of isolating a peptide which immunologically mimics a portion of CspA, comprising the steps of:
(1) identifying protective antibodies reactive with said CspA;
(2) contacting a phage-display library, having phage, with one or more of said protective antibodies identified in step 1;
(3) isolating one or more phage, having a displayed peptide, which bind one or more of the protective antibodies; and
(4) selecting, for all said phage isolated in step 3, the peptides or peptide fragments to which the antibodies have bound.

A peptide which “immunologically mimics” CspA is a substance that elicits an antibody response against the CspA. The peptide is preferably greater than five amino acids, although peptides of any length are within the scope of the invention.

The protective antibodies within the invention are antibodies shown to protect against GBS infection or to ameliorate the effects of a GBS infection. Any bactericidal assay that is known in the art may be used to identify the protective antibodies of the invention. In addition, protective antibodies can be identified by use of the lethal challenge assay in which laboratory animals, generally mice, are injected with a lethal amount of the bacteria being tested. Antibodies are then administered and mouse survival is determined. Those antibodies that are able to protect against death are considered to be protective.

“Contacting”, as used herein, refers to incubation for a sufficient period of time to permit antibody/antigen binding to occur, as can be easily measured by methods routine in the art.

“Phage display library”, as used herein, refers to a multiplicity of phage which express random amino acid sequences of between 7 and 15 amino acids at a location which may be bound by an antibody.

Particularly preferred in the practice of this invention is a phage display library produced by the method of Burritt et al. (Burritt, J. B. et al. 1996 Anal. Biochem. 238:1). Phage has the usual meaning it is given by one of ordinary skill in the art. (See, for example, Sambrook, J. E. et al. 1989 Molecular Cloning: a Laboratory Manual Cold Spring Harbor, N.Y. Cold Spring Harbor Laboratory)

The phrase “antibodies specific for CspA” refers to monoclonal or polyclonal antibodies which bind the substance CspA with an affinity greater than the average unselective affinity which these antibodies show for substances structurally unrelated to CspA. Antibodies of all isotypes, i.e., IgG, IgA, IgM, IgD, and IgE, may be used.

The phrase “isolating one or more phage . . . which bind one or more of the antibodies” means physically removing from the phage display library phage that bind the antibody with greater affinity than that between the antibody and a structurally unrelated antigen.

Although any technique for isolation may be used in the practice of the invention, a preferred method is affinity purification, which is well known in the art. A particularly preferred means of isolating phage from the phage display library is to first preabsorb the library repeatedly with cyanogen bromide activated sepharose 4B beads coated with antibodies specific for an antigen structurally unrelated to CspA. Following repeated preabsorption, the library is incubated overnight with cyanogen bromide activated sepharose 4B beads coated with one or more antibodies specific for CspA. Following this incubation, these same beads are washed extensively, and then eluted into two separate pools, the first pool being formed by elution with 0.1M glycine pH 2.2, with the second being formed by elution with 0.5M NH₄ OH pH 7. Next, the eluated pools are amplified, most preferably in an E. coli strain. Following elution, each pool is reapplied to the column, and the same incubation and washing procedure employed, eluting (in the end) with the same substance used to generate the original pool. This process is repeated for each pool, most preferably three times. Finally, phage are tested for binding to the selecting antibody using immunoblots of plaques. Phage which pass this final round are cloned, amplified to high titer, and purified by precipitation, most preferably with 2.5% polyethylene glycol.

“Displayed peptide” refers to peptides having an amino-acid sequence of between 7 and 15 amino acids which varies randomly between each of the individual phage which make up the phage display library.

Once the peptides are isolated by this process, they may be used in a vaccine. The vaccine may include the peptide itself or the peptide may be conjugated to a carrier or otherwise compounded. Carrier preferably refers to a T-dependent antigen which can activate and recruit T-cells and thereby augment T-cell dependent antibody production. However, the carrier need not be strongly immunogenic by itself, although strongly immunogenic carriers are within the scope of this invention. Multiple copies of the carrier are also within the scope of this invention. Multiple copies of the peptide are also within the scope of the invention, either unconjugated or conjugated to one or more copies of the carrier. Fragments and derivatives of the peptide are also within the scope of the invention, either alone or in combination with each other, not necessarily identically reproduced, and either unconjugated or conjugated to one or more copies of the carrier. Fusion proteins containing single or multiple copies of the peptide or parts thereof are also within the scope of the invention. In a further embodiment, microbes that express the DNA of the fusion protein are within the invention. In yet another embodiment, DNA encoding any and all of these substances is within the scope of the invention.

In a preferred embodiment, the carrier is a protein, a peptide, a T cell adjuvant or any other compound capable of enhancing the immune response. The protein may be selected from a group consisting of but not limited to viral, bacterial, parasitic, animal and fungal proteins. In a more preferred embodiment, the carrier is albumin (such as bovine serum albumin (BSA)), keyhole limpet hemocyanin (KLH), ovalbumin (OVA), tetanus toxoid, diphtheria toxoid, or bacterial outer membrane protein, all of which may be obtained from biochemical or pharmaceutical supply companies or prepared by standard methodology (Cruse, J. M. et al. 1989 Contrib. Microbiol. Immunol. 10:1). Other proteins that could function as carriers would be known to those of ordinary skill in the art of immunology.

The isolated peptides, with or without further compounding, may be immunogenic or, alternatively, the immunogenicity may arise from the compounding. Methods of measuring immunogenicity are well known to those in the art and primarily include measurement of serum antibody including measurement of amount, avidity, and isotype distribution at various times after injection of the construct. Greater immunogenicity may be reflected by a higher titer and/or increased life span of the antibodies. Immunogenicity may also be measured by the ability to induce protection to challenge with noxious substance or organisms. Immunogenicity may also be measured using in vitro bactericidal assays as well as by the ability to immunize neonatal and/or immune defective mice. Immunogenicity may be measured in the patient population to be treated or in a population that mimics the immune response of the patient population.

A particularly preferred means of determining the immunogenicity of a given substance is to first obtain sera of mice both before and after immunization with the substance. Following this, the strength of the post-immunization sera binding to CspA is ascertained using an ELISA, and compared against the ELISA results obtained for the pre-immunization sera.

Pharmaceutically acceptable carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical carriers are described in Martin, E. W., Remington's Pharmaceutical Sciences, specifically incorporated herein by reference. These carriers can also contain immunoadjuvants, including but not limited to alum, aluminum compounds (phosphate and hydroxide), and muramyl dipeptide derivatives.

The invention also relates to the treatment of a patient by administration of an immunostimulatory amount of the vaccine. Patient refers to any subject for whom the treatment may be beneficial and includes mammals, especially humans, horses, cows, dogs, and cats as well as other animals, such as chicks. An immunostimulatory amount refers to that amount of vaccine that is able to stimulate the immune response of the patient for the prevention, amelioration, or treatment of diseases. Of course, as noted above, the immunostimulation may result from the form of the antibody or the adjuvant with which it is compounded.

The vaccine of the invention may be administered by any route, but is preferably administered topically, mucosally or orally. Other methods of administration will be familiar to those of ordinary skill in the art, including intravenous, intramuscular, intraperitoneal, intracoporeal, intrarticular, intrathecal, intravaginal, intranasal, oral and subcutaneous injections.

### EXAMPLE 1

Introduction

We performed experiments to determine whether rabbit antibody raised to recombinant CspA protein could confer passive immunity to neonatal rats subsequently challenged with GBS.

Methods

Antibody to CspA was generated by immunizing a New Zealand white rabbit with the recombinant CspA-glutathione S-transferase fusion protein expressed and purified as described above and by subsequently exsanguinating the rabbit. Passive immunization of 24-48 hour old Sprague-Dawley rat pups was accomplished by administering 50 μl of anti-CspA heat-inactivated hyperimmune serum (neat, diluted ⅕, 1/10, and 1/20 in PBS) via intraperitoneal injection. As a control, 50 μl of normal rabbit preimmune serum was administered. After injection, the pups were returned to their mothers for 2 hours to allow for peak circulation of the sera. The highly virulent, type III GBS isolate COH1, was used as a challenge strain. COH1 was grown to an OD₆₀₀ (optical density) of 0.6, washed once in a volume of phosphate-buffered saline (PBS) equal to the original culture volume, harvested, then resuspended to a final OD₆₀₀ of 0.35 (approximately 1×10⁸ CFU/ml) in PBS. The suspension was diluted by 1/1000, and 50 μl (approximately 1870 bacteria) of the suspension were used to challenge the rat pups via subcutaneous injection. The pups were monitored for death for 72 hours. At 36 hours, two animals of each test group were sacrificed and the spleens were removed and homogenized in PBS. Dilutions of the resulting suspension were plated to Todd-Hewitt agar to test for the presence of GBS, and to verify that no contamination was present. The entire experiment was conducted twice, once in which dilutions of the hyperimmune serum were performed, and once where dilutions were not performed.

Results

In the first experiment, using undiluted hyperimmune serum, 12 of 20 rats died in the group that was vaccinated with anti-CspA serum, and 19 of 20 died in the control group that was vaccinated with normal rabbit antiserum (preimmune sera from same rabbit). In the second experiment, where dilutions of the hyperimmune serum were prepared, 9 of 14 animals treated with neat antiserum died, 7 of 14 animals treated with a ⅕ dilution of antiserum died, 6 of 14 animals treated with a 1/10 dilution of antiserum died, 8 of 14 animals treated with a 1/20 dilution of antiserum died, and 12 of 14 animals treated with normal rabbit antiserum died.

### CONCLUSION

In both experiments, passive immunization with anti-CspA serum provided protection against GBS challenge. In the experiment in which dilutions of the antiserum were administered, the animals that received treatment with the ⅕ dilution of anti-CspA serum displayed the greatest protection from challenge with GBS. In conclusion, these experiments indicate that anti-CspA-GST serum was protective in neonatal rats against subsequent lethal GBS challenge.

### EXAMPLE 2

Introduction

The mechanism of protection against GBS infection in neonates depends on the transplacental transfer of protective IgG immunoglobulins from the mother (Lin, F. C. et al. 2001 J Infect Dis 184:1022-1028). Therefore a candidate GBS vaccine must be capable of raising an IgG response. The corresponding IgG antibody can then be tested in animal passive protection models for the ability to confer protection against challenge with a lethal dose of GBS.

The CSP protein was expressed, purified and used to raise rabbit anti-sera as described above in Example 1. The IgG antibody was then purified using Protein A affinity chromatography. The purified IgG from anti-sera raised against the Group B Streptococcus (GBS) protein CSP(CSP) were then tested on two occasions in a rat pup protection model. Purified IgG from anti-sera raised against whole cell (WC) GBS were used as the positive control.

Methods

Bacterial Strains

The GBS strain (type 1a/c A909) used as a challenge strain in these studies was obtained from the National Collection of Type Cultures (NCTC reference number 11078, batch number 3).

Animals

Pregnant female Sprague Dawley rats, aged about 70 days, were obtained from Charles River (Margate, Kent, UK). On arrival, animals were housed in individual ventilated cages, lined with Gold Flake wood chip bedding (Lillico Attlee, Aylesford, Kent), at 21±1° C. with a relative humidity of 35-55% under a 12-hour dark/light cycle (lights on at 08:00). All animals had ad libitum access to tap water and RM3P food (Special Diet Services Ltd., Witham, Essex, UK). The animals were housed and maintained in accordance with the Code of Practice for the Housing and Care of Animals used in the Scientific Procedures, as issued by the UK Home office. All experiments were carried out under the authority of a project license granted under the Animals (Scientific Procedures) Act 1986.

IgG Purification from the Rabbit Anti-Sera Raised Against Candidate Proteins.

Rabbit anti-sera were raised against CSP protein as described above in Example 1. In all cases IgG was purified using Protein A affinity chromatography using a standard procedure.

Protective Effects of Purified IgG Anti-Sera Against Lethal Challenge with GBS in the Refined Rat Pup Protection Model

The purified IgG samples were tested using the rat pup protection model. Between 5-7 hours after intraperitoneal immunization with 50 μL purified IgG or PBS as a negative control, approximately 5×10⁴ CFU/50 μL of GBS were administered to each pup subcutaneously. The actual dose of bacteria administered in each experiment was determined by viable counts. The rat pups were monitored for 63-68.5 hours after GBS challenge and animals showing signs of extreme discomfort were sacrificed. The end-point criteria are described below. PBS was used as the negative control because in previous experiments pre-immune control serum was shown to have no effect on the survival of rat pups.

The number of rat pups sacrificed and their time of death were recorded.

Animal Model Endpoint Criteria

Primary Check Clinical Signs:

The criteria used to determine end point were as follows and comprise part of the project license:
Redness at site of inoculation.
Generalized erythema.
Subcutaneous haemorrhage.
Pallor of the head and extremities.
Reduced body temperature (cold to touch.)
   Secondary Check Clinical Signs:
Increased muscle tone/convex curvature to back.
Substantial subcutaneous haemorrhage.
Development of any of the primary check signs was taken as an indicator of developing poor health, resulting in an increased frequency of checks. When either of the secondary check signs were noted in addition to 2 or more of the primary check signs the end point was determined as having been reached and the animal was culled.
   Quantification of the Actual Challenge GBS Dose given to the Rat Pups in each Study

For each study, the dose of GBS administered to the pups in a 50 μL volume was calculated from dilutions of inoculum spread onto blood agar plates. Colony counts were recorded after incubation of the plates overnight at 37° C. and the results summarized. In all cases, the dose of GBS administered was higher than the nominal 5×10⁴ CFU in a 50 μL volume (Table 2).

TABLE 2
The number of GBS CFUs administered to each
pup in the animal studies presented. The CFUs
were calculated from the number of colonies
counted on blood agar plates at serial dilutions
of either approximately 1 × 10² or 1 × 10³ CFU/mL.

| Study | Number of GBS cells administered |
|---|---|
| Number | per pup per 50 μL dose. |
| 1 | 6.8 × 10⁴ |
| 2 | 9.5 × 10⁴ |
| 5 | 9.2 × 10⁴ |
| 6 | 8.5 × 10⁴ |

Results
The Effect on Rat Pups of Challenge with the A909 Strain of GBS at Approximately 5×10⁴ CFUs in 50 μL (study 1 and study 2)

In these studies, the effects of challenging rat pups with the A909 strain of GBS were assessed. The GBS dose was administered to each rat pup subcutaneously. The pups were monitored for up to 68.5 hours after GBS challenge and animals showing signs of extreme distress, as described above, were sacrificed.

TABLE 3
The number of rat pups that were sacrificed after
challenge with the A909 strain of GBS. The number
of pups that survived was monitored until 64 hours
after challenge in study 1 and 68.5 hours in study 2.

| | | Total no. of | Total no. of pups | % pups that |
|---|---|---|---|---|
| Study | Dose | rat pups | sacrificed | survived challenge |
| 1 | 6.8 × 10⁴ | 28 | 22 | 21 |
| 2 | 9.5 × 10⁴ | 26 | 20 | 23 |
| 2 | 9.5 × 10⁴ | 27 | 26 | 4 |

The results from these studies show that the percentage of pups that survived after challenge with A909 GBS at a dose of approximately 5×10⁴ CFUs was between 4% and 23%. These results indicate variability in survival rate in experiments conducted with the same challenge dose on the same occasion.

The Protective Effects of Purified CSP and WC GBS IgG Against GBS Challenge (Study 5)

In this study, the protective effects of IgG purified from anti-sera raised against CSP and whole cell (WC) A909 GBS were compared to PBS solution in rat pups challenged with the A909 strain of GBS. The number of sacrificed animals was recorded at the various time intervals, as shown in table 4 below, and expressed as a percentage of the total number of pups in each group.

TABLE 4
The number of rat pups that were sacrificed following inoculation with either PBS solution
(negative control) or the purified IgG anti-sera against WC A909 (positive control), and
CSP (test antisera), 5-7 hours before challenge with 9.2 × 10⁴ CFU/50 μL
of the A909 strain of GBS. The number of pups that survived was monitored
over a 63-hour period following the GBS challenge.

| Protein to | | | | | | | | | | | | |
|---|---|---|---|---|---|---|---|---|---|---|---|---|
| which | Total | | | | | | | | | | | |
| purified | no. of | Number of rat pups that were sacrificed 14-63 hours | | | | | | | | | Total no. of pups | % pups that |
| IgG was | rat | after GBS challenge | | | | | | | | | sacrificed after 63 | survived after 63 |
| raised | pups | 14 | 15.5 | 18.5 | 21.5 | 23.5 | 39 | 42 | 45.5 | 63 | hours | hours |
| PBS | 30 | 7 | 3 | 5 | 3 | 1 | 0 | 0 | 0 | 0 | 19 | 37 |
| WC GBS | 30 | 0 | 0 | 0 | 0 | 0 | 0 | 0 | 0 | 0 | 0 | 100 |
| CSP | 30 | 4 | 4 | 0 | 0 | 0 | 5 | 1 | 0 | 0 | 14 | 53 |

The results from this study show that 37% of the rat pup group receiving no protective immunization (PBS solution negative control group) survived. The percentage survival in groups administered IgG purified from anti-GBS whole cell sera (positive control) was 100%. The percentage survival in groups administered IgG purified from anti-Csp sera was 53%.

The Protective Effects of Purified CSP and WC GBS IgG Against GBS Challenge (Study 6)

In this study, the protective effects of IgG purified from anti-sera raised against CSP and whole cell A909 GBS (WC) were compared to PBS. The IgG purified from anti-sera raised against whole cell A909 was tested at a 1 in 5 dilution to assess levels of protection in comparison to the non-diluted IgG. There were 30 rat pups in each experimental group, and the number of rat pups sacrificed during the 63-hour period after inoculation with GBS was recorded. The sacrificed animals were categorized into the various time intervals, as seen in table 5, collated, and expressed as a percentage of the total number of pups used in each experimental group.

TABLE 5
The number of rat pups that were sacrificed following inoculation with either PBS
solution (negative control) or the purified IgG anti-sera against 1:5 WC A909,
WC A909, and CSP, 5-7 hours before challenge with 8.5 × 10⁴ CFU/50 μL
of the A909 strain of GBS. The number of pups that survived was monitored
over a 63-hour period following the GBS challenge.

| Protein to | | | | | | | | | | | | | Total no. of | % pups |
|---|---|---|---|---|---|---|---|---|---|---|---|---|---|---|
| which | Total | | | | | | | | | | | | pups | that |
| purified | no. of | Number of rat pups that were sacrificed | | | | | | | | | | | sacrificed | survived |
| IgG was | rat | 15-63 hours after GBS challenge | | | | | | | | | | | after 63 | after 63 |
| raised | pups | 15 | 16.5 | 19 | 22.5 | 24 | 39 | 41 | 43.5 | 45.5 | 47.5 | 63 | hours | hours |
| PBS | 30 | 11 | 3 | 3 | 2 | 2 | 2 | 1 | 0 | 0 | 0 | 0 | 24 | 20 |
| 1:5 WC GBS | 30 | 0 | 0 | 0 | 0 | 0 | 2 | 0 | 0 | 0 | 0 | 0 | 2 | 93 |
| WC GBS | 30 | 2 | 0 | 0 | 0 | 0 | 0 | 0 | 0 | 0 | 0 | 0 | 2 | 93 |
| CSP | 30 | 4 | 1 | 1 | 0 | 1 | 2 | 0 | 0 | 0 | 0 | 0 | 9 | 70 |

The results from this study show that the percentage survival in the rat pups that received PBS was 20% and the percentage survival in the groups receiving anti-WC IgG, both at a neat concentration and diluted 1 in 5 with PBS, was 93%. The percentage survival produced with the anti-CSP protected 70% of the animals within the group.

Statistical Analysis of Csp Data

A generalized linear model was fitted to the numbers of pups surviving in each group in each experiment, assuming a total of 30 pups tested on each occasion. The Genstat statistical package was used to fit the model, assuming a binomial error structure and logit link. A comparison was made of the odds of survival between the Csp treated group and the negative control PBS treated group. The results of this analysis gave an odds ratio estimate of 5.6 with 95% confidence limits of 2.7 to 11.9, which is statistically significant at the 5% significance level. This means that the odds of survival in the CSP treated group are 5.6 times greater than the odds of survival in the negative control group and that this ratio lies between 2.7 and 11.9 with 95% confidence.

Discussion

Killed whole cell GBS preparations have limited utility as vaccine candidates, as the majority of the protective immune responses they elicit are serotype specific. In contrast the CSP antigen is encoded by all GBS strains tested to date. These studies have shown that purified IgG raised against the CSP protein has a statistically significant protective effect in the passive protection rat pup model of GBS disease.

While the present invention has been described in some detail for purposes of clarity and understanding, one skilled in the art will appreciate that various changes in form and detail can be made without departing from the true scope of the invention. All figures, tables, appendices, patents, patent applications and publications, referred to above, are hereby incorporated by reference.

1. A vaccine comprising a pharmaceutically effective amount of a polypeptide sequence comprising an amino acid sequence encoding a CspA protease of Group B Streptococcus having SEQ ID NO: 2 or analogue, homologue, derivative, immunologically related polypeptide or fragment thereof containing at least one immunogenic epitope, which amount is effective to prevent or ameliorate the severity of Group B Streptococcus or related bacterial infection in a susceptible mammal in combination with a pharmaceutically acceptable vehicle.

2. The vaccine of claim 1 which further comprises an adjuvant.

3. The vaccine of claim 1 wherein the mammal is human.

4. The vaccine of claim 1 wherein the mammal is cow.

5. The vaccine of claim 1, wherein said CspA protease of Group B Streptococcus having SEQ ID NO: 2 or analogue, homologue, derivative, immunologically related polypeptide or fragment thereof is pre-pro domain or analogue, homologue, derivative, fragment or polymeric form thereof.

6. The vaccine of claim 1, wherein said CspA protease of Group B Streptococcus having SEQ ID NO: 2 or analogue, homologue, derivative, immunologically related polypeptide or fragment thereof is protease domain or analogue, homologue, derivative, fragment or polymeric form thereof.

7. The vaccine of claim 1, wherein said CspA protease of Group B Streptococcus having SEQ ID NO: 2 or analogue, homologue, derivative, immunologically related polypeptide or fragment thereof is A domain or analogue, homologue, derivative, fragment or polymeric form thereof.

8. The vaccine of claim 1, wherein said CspA protease of Group B Streptococcus having SEQ ID NO: 2 or analogue, homologue, derivative, immunologically related polypeptide or fragment thereof is cell-wall spacer domain or analogue, homologue, derivative, fragment or polymeric form thereof.

9. The vaccine of claim 1, wherein said CspA protease of Group B Streptococcus having SEQ ID NO: 2 or analogue, homologue, derivative, immunologically related polypeptide or fragment thereof is cell-wall anchor domain or analogue, homologue, derivative, fragment or polymeric form thereof.

10. A vaccine comprising a pharmaceutically effective amount of a polynucleotide sequence comprising a DNA sequence encoding a polypeptide sequence comprising an amino acid sequence encoding a CspA protease of Group B Streptococcus having SEQ ID NO: 2 or analogue, homologue, derivative, immunologically related polypeptide or fragment thereof containing at least one immunogenic epitope, which amount is effective to prevent or ameliorate the severity of Group B Streptococcus or related bacterial infection in a susceptible mammal in combination with a pharmaceutically acceptable vehicle.

11. The vaccine of claim 10, wherein the DNA sequence is taken from SEQ ID NO: 1.

12. A polynucleotide sequence comprising a DNA sequence encoding a polypeptide sequence comprising an amino acid sequence encoding a CspA protease of Group B Streptococcus having SEQ ID NO: 2 or analogue, homologue, derivative, immunologically related polypeptide or fragment thereof containing at least one immunogenic epitope, wherein the DNA sequence is taken from SEQ ID NO: 1.

13. A purified polynucleotide sequence comprising an oligomer having a sequence complementary to at least about 6 contiguous nucleotides of cspA having SEQ ID NO: 1.

14. A probe comprising the purified polynucleotide sequence of claim 13.

15. A primer comprising the purified polynucleotide sequence of claim 13.

16. A method of protecting a susceptible mammal against Group B Streptococcus or related bacterial infection comprising administering to the mammal a pharmaceutically effective amount of a vaccine of any of claims 1-11.

17. A method for the production of CspA protease, immunologically related polypeptides, and fragments thereof, comprising the steps of
(a) culturing a unicellular host organism transfected with a vector containing a DNA sequence coding for said polypeptide or fragment and one or more expression control sequences operatively linked to the DNA sequence, and
(b) recovering a substantially pure polypeptide or fragment.

18. Purified vector of claim 17.

19. Unicellular host organism of claim 17.

20. Purified anti-CspA antibody that is immunologically reactive with CspA or specifically binds CspA.

21. A method of protecting a susceptible mammal against Group B Streptococcus or related bacterial infection comprising administering to the mammal a pharmaceutically effective amount of a purified anti-CspA antibody, which amount is effective to prevent or ameliorate the severity of Group B Streptococcus or related bacterial infection in a susceptible mammal.

22. A method for the detection of Group B Streptococcus or related bacteria in a biological sample comprising the steps of:
(a) isolating the biological sample from a patient;
(b) incubating a purified anti-CspA antibody, or fragment thereof, with the biological sample to form a mixture; and
(c) detecting specifically bound antibody or fragment in the mixture which indicates the presence of Group B Streptococcus or related bacteria.

23. A method for the detection of antibodies specific to Group B Streptococcus or related bacteria in a biological sample comprising:
(a) isolating the biological sample from a patient;
(b) incubating a polypeptide having SEQ ID NO: 2, or fragment thereof, with the biological sample to form a mixture; and
(c) detecting specifically bound polypeptide in the mixture which indicates the presence of antibodies specific to Group B Streptococcus or related bacteria.

24. A method for the detection of Group B Streptococcus or related bacteria in a biological sample comprising the steps of:
(a) isolating the biological sample from a patient;
(b) incubating a DNA probe having SEQ ID NO: 1, or fragment thereof, with the biological sample to form a mixture; and
(c) detecting specifically bound DNA probe in the mixture which indicates the presence of Group B Streptococcus or related bacteria.

25. An attenuated Group B Streptococcus microorganism comprising a mutation in cspA gene that disrupts the expression of CspA protease.

26. A vaccine comprising a pharmaceutically effective amount of the attenuated Group B Streptococcus microorganism of claim 25, which amount is effective to prevent or ameliorate the severity of Group B Streptococcus infection in a susceptible mammal in combination with a pharmaceutically acceptable vehicle.

27. A method of protecting a susceptible mammal against Group B Streptococcus infection comprising administering to the mammal a pharmaceutically effective amount of the vaccine of claim 26.

28. A method of isolating a peptide which immunologically mimics a portion of CspA comprising the steps of:
(a) identifying protective antibodies reactive with said CspA;
(b) contacting a phage-display library, having phage, with one or more of said protective antibodies identified in step a;
(c) isolating one or more phage, having a displayed peptide, which bind one or more of the protective antibodies; and
(d) selecting, for all said phage isolated in step c, the peptide or peptide fragments to which the antibodies have bound.

29. A mimetic peptide that induces an immunoprotective response against GBS, wherein said peptide specifically binds to an antibody that specifically binds to CspA.

30. A vaccine comprising a mimetic peptide that induces an immunoprotective response against GBS, wherein said peptide specifically binds to an antibody that specifically binds to CspA; and a pharmaceutically acceptable carrier.

31. A method of treating a patient comprising administering to the patient an immunostimulatory amount of a vaccine comprising: a mimetic peptide that induces an immunoprotective response against GBS, wherein said peptide specifically binds to an antibody that specifically binds to CspA; and a pharmaceutically acceptable carrier.
